(19) United States
(12) Patent Application Publication
(10) Pub. No.: US 2026/0007303 A1
(43) Pub. Date: Jan. 8, 2026

(54) AN ENDOSCOPE COMPRISING A SHOVEL

(71) Applicant: MICROSTEER LTD, Nazareth (IL)

(72) Inventors: Adi STRAUSS, Alonei Abba Village (IL); Ofer PILLAR, Kiryat Haim-Haifa (IL); Eldar COHEN, Harish (IL); Tzahi BESSENOV, Haifa (IL)

(21) Appl. No.: 18/881,125

(22) PCT Filed: Jul. 4, 2023
(86) PCT No.: PCT/IL2023/050690

§ 371 Date: Jan. 3, 2025

Related U.S. Application Data
(60) Provisional application No. 63/358,181, filed on Jul. 4, 2022.

(51) Int. Cl. A61B 1/00

(52) U.S. Cl. CPC A61B 1/00087

(57) ABSTRACT

The present subject matter provides a shovel configured to attach to a distal edge of an endoscope and protrude from a lower part of the distal edge, and an endoscope comprising the shovel attached to a distal edge of the endoscope. Additional embodiments of the shovel and the endoscope comprising the shovel are disclosed herein.

### CROSS-REFERENCE TO RELATED APPLICATION

This application claims priority to U.S. Provisional Patent Application No. 63/358,181, filed Jul. 4, 2022, the entire contents of which is incorporated herein by reference in its entirety.

### FIELD

The present subject matter relates to endoscopes. More particularly, the present subject matter relates to endoscopes comprising a shovel.

### BACKGROUND

Dissection and removal of tissues, for example tumors, tissues suspected to be tumors, like polyps, and the like, from a patient's organs, is a procedure known in the art. For the sake of simplicity, a tissue to be manipulated is termed hereinafter “target tissue”. Manipulation of target tissues, particularly target tissues that are slightly convex, slightly concave and/or slightly distinct from a surface of a tissue, and more particularly manipulation of large tissues, is challenging, particularly when the target tissues reside in a cavity in the body. For example, in order to dissect, or cut, a target tissue, the target tissue has to be elevated. Therefore, manipulation in general, and dissection, or cutting, in particular, of soft target tissues, is challenging, particularly when there is a desire to manipulate the target tissue by less invasive procedures, like endoscopic procedures, polypectomy, and the like. The manipulation of the target tissue is also challenging when the target tissue is non-symmetric.

Several types of manipulation of the target tissue are challenging, for example close imaging of all the sides of the target tissue, injection of substances into all the sides of a submucosal layer of the target tissue, dissecting and ablation of all sides of the target tissue, for example by polypectomy and/or ablation and/or tissue disconnecting, combinations thereof and the like.

### SUMMARY

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

According to one aspect of the present subject matter, there is provided a shovel configured to attach to a distal edge of an endoscope and protrude from a lower part of the distal edge.

According to another aspect of the present subject matter, there is provided an endoscope comprising the shovel, attached to a distal edge of the endoscope.

According to one embodiment, the shovel has a substantially concave structure.

According to one embodiment, the shovel further comprising a connector configured to connect the shovel to the distal edge of the endoscope.

According to one embodiment, the shovel further comprising a shovel rail that runs along a width of the shovel and is substantially vertical relative to a length of the endoscope, wherein the shovel rail is configured to allow a vehicle to move along the shovel rail, thus allowing the vehicle to move along a width of the shovel.

According to one embodiment, the endoscope further comprising a gripper attached to a distal edge of the endoscope, wherein the gripper is configured to grip a target tissue in a vicinity of the shovel.

According to one embodiment, the shovel further comprising a shovel cover comprising a covering element configured to cover the shovel from above and allow protrusion of a tool out from the shovel.

According to one embodiment, the shovel cover comprising an opening at a front side relative to the distal edge of the endoscope, the opening is configured to allow protrusion of a tool therethrough.

According to one embodiment, the shovel cover permanently covers the shovel.

According to one embodiment, the shovel covert is removable.

According to one embodiment, a cover connector is attached to the shovel cover and is configured to connect the shovel cover to the distal edge of the endoscope.

According to one embodiment, the cover connector is in a form of a sleeve that engages with the distal edge of the endoscope.

According to one embodiment, the cover connector is configured to permanently connect with the distal edge.

According to one embodiment, the cover connector is configured to removably connect with the distal edge.

According to one embodiment, the covering element is at least partially foldable.

According to one embodiment, the shovel is axially attached to the connector with a horizontal axis positioned at a bottom part of the connector, wherein the horizontal axis allows swiveling of the shovel either upwards, or downwards, or upwards and downwards.

According to one embodiment, the shovel is axially attached to the connector with a vertical axis positioned at a border between the shovel and the connector, wherein the vertical axis allows swiveling of the shovel either to the right, or to the left, or to the right and to the left.

According to one embodiment, the shovel further comprising a swiveling sub-shovel 403 positioned over, or under the shovel, and configured to swivel aside, thus allowing extension of the width of the shovel.

According to one embodiment, the shovel further comprising a multi-barreled cutting tool, wherein the multi-barreled cutting tool comprising a plurality of barrels arranged on the shovel.

According to one embodiment, the shovel further comprising a lifting element configured to lift a target tissue, wherein the lifting element is configured to be in an elevated state and in a lowered state.

According to one embodiment, the lifting element comprising a tissue engaging element configured to engage with the target tissue when lifting the target tissue.

According to one embodiment, the tissue engaging element has a high friction coefficient, thus allowing tight engagement of the tissue engaging element with the target tissue, and avoid slippage of the target tissue from the tissue engaging element during the lifting of the target tissue.

According to one embodiment, the shovel further comprising a sucking lifting element configured to engage with a target tissue by a sucking force and lift or move aside the engaged target tissue, wherein the sucking lifting element has a nozzle-like structure.

According to one embodiment, the sucking lifting element comprising a tissue sucking element positioned at a distal end of the nozzle-like structure of the sucking lifting element, wherein the sucking lifting element is hollow, and the tissue sucking element is an opening at the distal end of the sucking lifting element, wherein the tissue sucking element is configured to engage with the target tissue, and then a sucking action in the sucking lifting element causes a formation of a vacuum force in the tissue sucking element, causing the target tissue to be held by the sucking lifting element.

According to one embodiment, the sucking lifting element further comprising at least one bulge on a top side of the sucking lifting element, or on a bottom side of the sucking lifting element, or on both the top side and the bottom side of the sucking lifting element, wherein the at least one bulge allows engagement with the target tissue.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIG. 1 schematically illustrates, according to an exemplary embodiment, a surface tissue, and a target tissue.

FIG. 2 schematically illustrates, according to an exemplary embodiment, a perspective view of prior art endoscope approaching a target tissue.

FIG. 3A schematically illustrates, according to some exemplary embodiments, a distal edge of an endoscope comprising a shovel and a first mechanism for facilitating movement of a vehicle along a width of the shovel.

FIG. 3B schematically illustrates, according to some exemplary embodiments, a distal edge of an endoscope comprising a shovel and a second mechanism for facilitating movement of a vehicle along a width of the shovel.

FIGS. 4A-B schematically illustrate, according to some exemplary embodiments, a view from two different angles, of a distal edge of an endoscope comprising a shovel and a third mechanism for facilitating movement of a vehicle along a width of the shovel.

FIG. 5 schematically illustrates, according to an exemplary embodiment, two positions of an endoscope comprising a shovel dissecting a target tissue.

FIGS. 6A-B schematically illustrate, according to an exemplary embodiment, a perspective view of a collapsed state and an extended state, respectively, of a gripper attached to an endoscope comprising a shovel.

FIG. 7 schematically illustrates, according to an exemplary embodiment, another structural embodiment of a gripper and an arm.

FIG. 8 schematically illustrates, according to an exemplary embodiment, an endoscope comprising a shovel, a gripper and an arm further comprising a rear imaging device.

FIG. 9 schematically illustrates, according to an exemplary embodiment, a field of vision of a rear imaging device.

FIG. 10 schematically illustrates, according to an exemplary embodiment, a side view of an endoscope comprising a shovel imaging a rear view of a target tissue gripped with a gripper inside a cavity.

FIG. 11 schematically illustrates, according to an exemplary embodiment, a side view of an endoscope comprising a shovel and a gripper dissecting a target tissue gripped with a gripper.

FIG. 12 schematically illustrates, according to an exemplary embodiment, an endoscope comprising a shovel and a shovel cover.

FIGS. 13A-C schematically illustrate, according to an exemplary embodiment, various embodiments of size and shape of a shovel cover.

FIG. 14A schematically illustrates, according to an exemplary embodiment, a side perspective view of a foldable shovel cover in a spread state.

FIGS. 14B-C schematically illustrate, according to an exemplary embodiment, a side view of a foldable shovel cover in a spread state and a folded state, respectively.

FIG. 14D schematically illustrates, according to an exemplary embodiment, a side view of a folding shovel cover.

FIGS. 15A-B schematically illustrate, according to an exemplary embodiment, a side perspective view of a shovel swiveling upwards and a shovel swiveling downwards, respectively.

FIG. 16A schematically illustrates, according to an exemplary embodiment, a front perspective view of a shovel configured to swivel side to side.

FIG. 16B schematically illustrates, according to an exemplary embodiment, a side perspective view of a shovel configured to swivel to the right, or to the left, or to the right and to the left, dissecting a target tissue.

FIGS. 17A-B schematically illustrate, according to an exemplary embodiment, a front perspective view of a foldable shovel in a folded state and an open state, respectively.

FIGS. 18A-C schematically illustrate, according to an exemplary embodiment, a front perspective view of a shovel comprising a swiveling sub-shovel, in three states.

FIGS. 19A-B schematically illustrate, according to an exemplary embodiment, a front perspective view of an endoscope comprising a shovel and a multi-barreled cutting tool, and a side perspective close view of the shovel and the multi-barreled cutting tool, respectively.

FIG. 20 schematically illustrates, according to an exemplary embodiment, a front perspective view of an endoscope comprising a shovel and a multi-barreled cutting tool and a shovel cover.

FIG. 21 schematically illustrates, according to an exemplary embodiment, a side perspective view of an endoscope comprising a shovel and a multi-barreled cutting tool and a shovel cover, dissecting a target tissue.

FIG. 22 schematically illustrates, according to an exemplary embodiment, an endoscope comprising a circular shovel and a circular multi-barreled cutting tool, dissecting a target tissue.

FIG. 23 schematically illustrates, according to an exemplary embodiment, a side perspective view of an endoscope comprising a shovel and a lifting element.

FIGS. 24A-B schematically illustrate, according to an exemplary embodiment, a side perspective view of an endoscope comprising a shovel and a lifting element in a lowered state and an elevated state lifting a target tissue, respectively.

FIG. 25 schematically illustrates, according to an exemplary embodiment, a side perspective view of an endoscope comprising a sucking lifting element.

FIGS. 26A-B schematically illustrate, according to an exemplary embodiment, a side perspective view of an endoscope comprising a sucking lifting element engaging with a target tissue, at a lowered state and an elevated state, respectively.

FIG. 27 schematically illustrates, according to an exemplary embodiment, a system for allowing controlled access of a tool to all sides of an elevated tissue in a body of a patient.

FIG. 28 schematically illustrates, according to an exemplary embodiment, a rail surrounding an elevated tissue, the rail protrudes from an endoscope inserted into a cavity of a body of a patient.

FIG. 29 schematically illustrates, according to an exemplary embodiment, a rail exiting a multi-lumen approaching an elevated tissue.

FIG. 30A schematically illustrates, according to an exemplary embodiment, a perspective view of a rail comprising a track and multiple ties extending from the track.

FIG. 30B schematically illustrates, according to an exemplary embodiment, an enlarged view of the track shown in FIG. 5A.

FIG. 30C schematically illustrates, according to an exemplary embodiment, an enlarged view of the track and ties shown in FIG. 5A.

FIG. 31 schematically illustrates, according to an exemplary embodiment, a perspective view of a rail comprising a track and multiple ties extending from the track exiting from an endoscope and surrounding an elevated tissue, while a vehicle stands on, or moves along, the rail and cuts the elevated tissue.

FIG. 32 schematically illustrates, according to an exemplary embodiment, an upper view of a rail comprising a track and multiple ties extending from the track, exiting an endoscope, and having various stages of extension.

FIG. 33 schematically illustrates, according to an exemplary embodiment, a side view of a tool comprising a wrap.

FIG. 34 schematically illustrates, according to an exemplary embodiment, an upper view of a cutting tool comprising a dynamic tissue remover.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

The present subject matter provides a shovel configured to attach to a distal edge of an endoscope. The present subject matter further provides an endoscope comprising a shovel. The endoscope comprising the shovel is used for manipulating tissues, preferably target tissues. The endoscope comprising a shovel has several advantages over prior art endoscopes. For example, the shovel stabilizes tools that are used during manipulation of target tissues with the endoscope comprising a shovel. In another example, the shovel allows cutting of, or injecting to, for example, a larger section of a target tissue, without moving the endoscope aside, compared to prior art endoscopes. For example, a prior art endoscope allows cutting of, or injection to, a substantially 2-3 mm section of a target tissue without moving the endoscope, while an endoscope comprising a shovel allows cutting of, or injection to, much larger sections of the target tissue, for example up to substantially 50 mm, or more, without moving the endoscope. In addition, the addition of the shovel to the endoscope, when a vehicle carrying a tool is moving along a rail of the shovel, allows more stable and controlled manipulation, for example dissection, of a target tissue, as well as prevents un-controlled, random, movements of the vehicle and the tool. Additional advantages of the endoscope comprising a shovel are described herein throughout the description of the present subject matter.

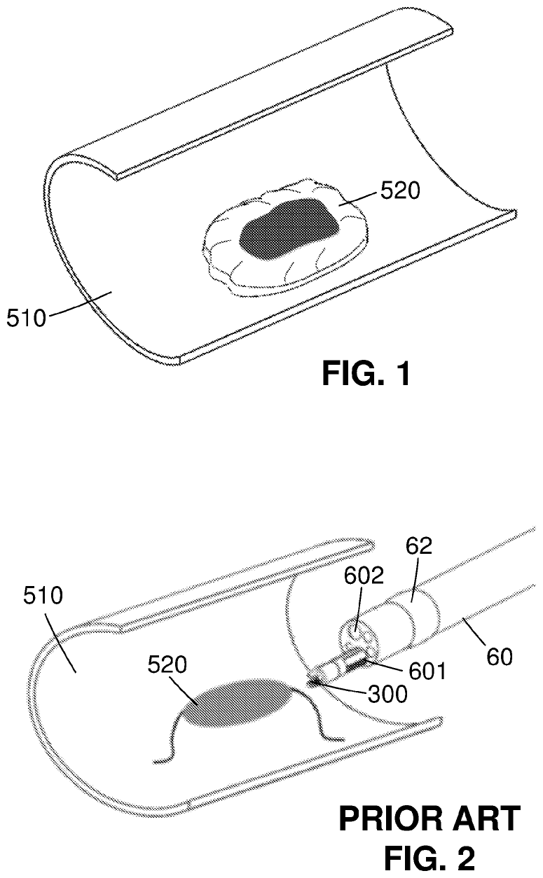

Referring now to FIG. 1, schematically illustrating, according to an exemplary embodiment, a surface tissue, and a target tissue. FIG. 1 illustrates an exemplary target tissue 520 extending from a surface tissue 510, or in other words, an elevated tissue 520. The surface tissue 510 is any type of tissue present in a body of a patient, for example a surface tissue 510 of an organ, or a cavity, in the body, like the colon, also known as the large intestine, as illustrated in FIG. 1, or any other type of tissue that can be accessed by any tool, for either surgical activity or any other manipulation.

The target tissue 520 is any type of tissue that there is a desire to manipulate it, for example dissect and separate the target tissue 520 from the surface tissue 510, and in some embodiments, remove the dissected and separated target tissue 520 from the body of the patient; close imaging of all the sides of the target tissue 520 from appropriate angles; injection of substances into all the sides of a submucosal layer of the target tissue 520; dissection and/or coagulation and/or ablation of all sides of the target tissue 520 according to needs that the clinical procedure requires, combinations thereof, and the like. The use of the term “sides” is intended also to all the curves that form the shape of the target tissue 520.

Some exemplary target tissues 520 include: tumors, tissues suspected to be tumors, like polyps, lesions, a combination thereof, and the like. The target tissue 520 can be either symmetric, or non-symmetric. The target tissue 520 can be hard or soft. The target tissue 520 can be a large and distinct tissue that can be easily manipulated, for example dissected and separated from the surface tissue 510. Alternatively, the target tissue 520 can be slightly elevated from the surface tissue 510, rendering its manipulation more challenging.

The present subject matter provides a shovel configured to attach to a distal edge of an endoscope. The present subject matter further provides an endoscope comprising a shovel. The endoscope comprising the shovel is used for the treatment of tissues, preferably target tissues 520.

According to one embodiment, the endoscope comprising a shovel is configured to allow access of various tools to a tissue, particularly a target tissue 520. Some exemplary tools include: a dissecting tool, a coagulating tool, an ablating tool, a grabbing tool, an imaging tool, an injecting tool and the like.

In some embodiments, the endoscope comprising a shovel of the present subject matter allows dissection and separation of a target tissue 520 from a surface tissue 510 in a body of a patient. In some other embodiments, the endoscope comprising a shovel of the present subject matter further allows removal of the dissected and separated target tissue 520 from the body of the patient. In some additional embodiments, the endoscope comprising a shovel of the present subject matter allows performance of additional manipulations on the target tissue 520 and its surroundings.

According to one embodiment, the shovel facilitates dissection of a target tissue 520. When a target tissue 520 is dissected, the shovel can enter into an incision in the dissected target tissue 520. This allows stabilization of the target tissue 520 during dissection, and allows an operator of the endoscope comprising a shovel to view the incision line and better control the dissection process.

According to one embodiment, the shovel allows stretching of the surface tissue 510 near a base of the target tissue 520. This embodiment allows an operator of the endoscope comprising a shovel to manipulate the target tissue 520 with enhanced stability and accuracy.

According to embodiment, the shovel is configured to prevent from a tool carried by the endoscope comprising a shovel to descent lower than the level of the shovel. This embodiment prevents damage by the tool to a healthy surface tissue 510 that is under the target tissue 520.

According to one embodiment, the shovel is configured to allow measurement of the target tissue, and any site that the shovel is adjacent to. This is because the shovel can be used as a length reference during imaging when the endoscope comprising a shovel is used.

According to one embodiment, the patient is an animal, particularly a vertebrate. According to another embodiment, the patient is a human.

The term “tool” as disclosed herein refers to any type of tool that is configured to be used during manipulation of tissues in a body of a patient. Some exemplary types of tools include; a dissecting tool configured to dissect a tissue; a grabbing tool configured to grab a piece of tissue; a storing tool configured to store an object, for example a piece of tissue, for example during removal of the object from the body of the patient; an imaging tool configured to acquire images inside a body of a patient; an illuminating tool configured to illuminate inside a body of a patient; an injecting tool configured to inject substances into a tissue; a burning tool configured to burn parts of a tissue, any combination thereof, and the like.

Referring now to FIG. 2, schematically illustrating, according to an exemplary embodiment, a perspective view of prior art endoscope approaching a target tissue. FIG. 2 illustrates an internal cavity having a surface tissue 510, and a target tissue 520 extending above the surface tissue 510. The prior art endoscope 60 that is inserted into the cavity comprises a distal edge 62. The distal edge 62 is the edge of the endoscope 60 that is inserted into the patient's body, and where at least one channel, for example channel 601 and channel 602, are opened in order to allow exit of tools transferred through the at least one channel. For example, FIG. 2 shows a cutting tool 300 that exits channel 601.

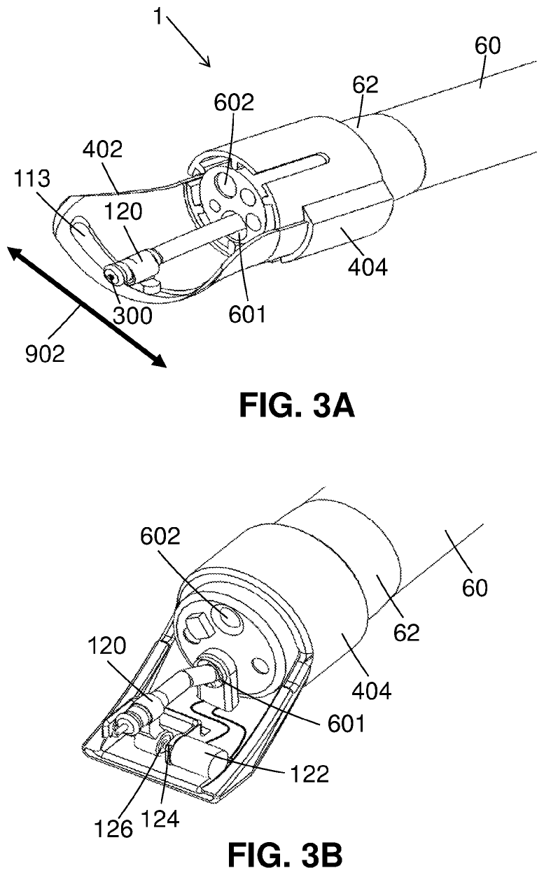

Referring now to FIG. 3A, schematically illustrating, according to some exemplary embodiments, a distal edge of an endoscope comprising a shovel and a first mechanism for facilitating movement of a vehicle along a width of the shovel. FIG. 3A shows a distal edge 62 of an endoscope 60. According to one embodiment, the endoscope 60 comprises a shovel 402 that is connected to the distal edge 62, or configured to connect to the distal edge 62 of the endoscope 60. The shovel 402 has a substantially concave structure and protrudes from a lower part of the distal edge 62. In other words, when the endoscope 60 moves over a surface tissue 510, the shovel 402 is configured to at least occasionally be in contact with the surface tissue 510.

According to one embodiment, a bottom surface of the shovel 402 is configured to slide over the surface tissue 510, for example during movement of the endoscope inside the cavity 550. This embodiment is advantageous over prior art endoscopes 60 that do not comprise a shovel 402 and therefore may face difficulties during their movement in the cavity 550.

Additional advantages of the shovel 402 relate to the manipulation of the target tissue 520, and are mentioned throughout the description hereinafter. Briefly, according to one embodiment, the shovel 402 is configured to flatten folds on the surface tissue 510 during the movement of the endoscope 60, thus facilitating smooth movement of the endoscope 60 comprising a shovel 402 inside the cavity 550. According to another embodiment, the shovel 402 is configured to push the target tissue 520, for example during manipulation of the target tissue 520. According to yet another embodiment, the shovel 402 is configured to protect the surface tissue 510 and the target tissue 520 against mistakes that may occur during manipulation of the target tissue 520. For example, the shovel 402 can prevent insertion of a cutting tool 300 to deep into the target tissue 520, or prevent penetration of the cutting tool 300 in a too low angle into the target tissue 520 in a manner that may damage areas of the tissue that are not of interest, and the like. In other words, the shovel 402 can prevent, or limit, no-desired movements of the endoscope 60 comprising a shovel 402, or of a tool, like a cutting tool 300, when manipulating a target tissue 520.

According to one embodiment, the shovel 402 is an integral part of the endoscope 60. According to this embodiment, the endoscope 60 is manufactured with a shovel 402 at the distal edge 62 of the endoscope 60. According to another embodiment, the shovel 402 is configured to be connected to a distal edge 62 of an endoscope 60. According to this embodiment, the shovel 402 comprises a connector 404 configured to connect the shovel 402 to the distal edge 62. Any mechanism of the connector 404 to connect to the distal edge 62 of the endoscope 60 is under the scope of the present subject matter. The exemplary connector 404 shown in FIG. 3A has a cylinder-like structure and is configured to be worn over the distal edge 62 and grasp the distal edge 62 of the endoscope 601. This embodiment allows installation of the shovel 402 on existing endoscopes 60.

According to one embodiment, the shovel 402 comprises a mechanism that facilitates movement of a vehicle 120 along a width of the shovel 402. This embodiment is advantageous over prior art endoscopes 60 because it allows a wider area of a target tissue 520 to be accessed by a tool, for example a cutting tool 300, compared to tools carried by prior art endoscopes. A first such mechanism is shown in FIG. 3A. Thus, according to one embodiment, the shovel 402 comprises a shovel rail 113 that runs along a width of the shovel 402. According to another embodiment, the shovel rail 113 is substantially vertical relative to a length of the endoscope 60. The shovel rail 113 is configured to allow a vehicle 120 to move along the shovel rail 113, thus allowing the vehicle 120 to move along a width of the shovel 402. The vehicle 120, shown in FIG. 3A, protrudes out of a channel 601 of the endoscope 60, and engages with the shovel rail 113. Arrow 902 shows the direction in which the vehicle 120 can move along the shovel rail 113, that is from side to side along the shovel rail 113, along the width of the shovel 402 and vertically to the length of the endoscope 60.

According to one embodiment, a tool, for example a cutting tool 300 is attached to the vehicle 120. Thus, according to the embodiment shown in FIG. 3A, the endoscope 60 comprising a shovel 402 is configured to dissect target tissues 520 by: inserting an endoscope 60 comprising a shovel 402 and a shovel rail 113 to a vicinity of a target tissue 520; inserting a vehicle 120 carrying a cutting tool 300 through the endoscope 60; engaging the vehicle 120 with the shovel rail 113, approaching the shovel 402 to a base of the target tissue 520; moving the vehicle 120 along the shovel rail 113 back and forth, as indicated with arrow 902 in FIG. 3A, while activating the cutting tool 300; and as the target tissue 520 is dissected by the cutting tool 300 pushing the endoscope 60 comprising the shovel 402 further toward the target tissue 520 to allow further dissection of the target tissue 520 by the cutting tool 300.

Referring now to FIG. 3B, schematically illustrating, according to some exemplary embodiments, a distal edge of an endoscope comprising a shovel and a second mechanism for facilitating movement of a vehicle along a width of the shovel. FIG. 3B shows a shovel 402 similar to the shovel 402 shown in FIG. 3A, comprising a second mechanism for facilitating movement of a vehicle along a width of the shovel. According to one embodiment, the second mechanism comprises, on the shovel 402, for example in a vicinity of a distal edge of the shovel 402, a screw 124 positioned along a width of the shovel 402, a bolt 126 attached to the vehicle 120 and screwed over the screw 124, and a motor 122 positioned substantially near a side of the shovel 402, wherein the screw 124 is attached to the motor 122 and the motor 122 is configured to rotate the screw 124. Since the motor 122 and the screw 124 are fixed in place, and the vehicle 120 to which the bolt 126 is attached are mobile, turning of the screw 124 by the motor 122 causes movement of the bolt 126 along the screw 124, thus causing movement of the vehicle 120 along the screw 124. Since the screw 124 is positioned along the width of the shovel 402, then turning to the screw 124 causes movement of the vehicle 120 along the width of the shovel 402. Turning of the screw 124 in a first direction, for example clockwise, causes the bolt 126 and the vehicle 120 to move, for example toward the motor 122, while turning of the screw 124 in a second direction, for example counterclockwise, causes the bolt 126 and the vehicle 120 to move, for example away from the motor 122.

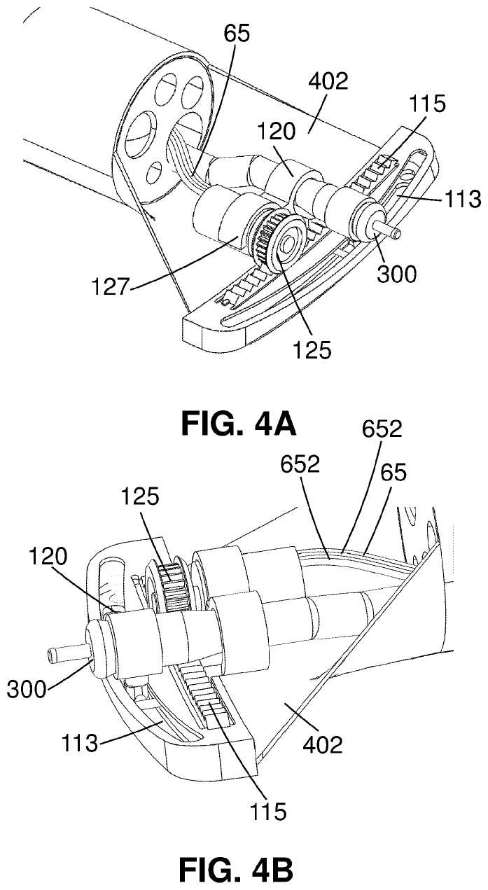

Referring now to FIGS. 4A-B, schematically illustrating, according to some exemplary embodiments, a view from two different angles, of a distal edge of an endoscope comprising a shovel and a third mechanism for facilitating movement of a vehicle along a width of the shovel. In the third mechanism, according to one embodiment, the vehicle 120 further comprises a wheel motor 127 attached to the vehicle 120 and a drive wheel 125 attached to the wheel motor 127, rotated by the wheel motor 127 and configured to drive the vehicle 120 during rotation. In other word, the drive motor 127 is configured to rotate the drive wheel 125 either clockwise, or counterclockwise. In addition, the shovel 402 further comprises a wheel rail 115 that corresponds to the drive wheel 125 of the vehicle 120 and runs along the width of the shovel 402. Thus, the drive wheel 125 is configured to move along the wheel rail 115 in correspondence to the direction of rotation of the drive wheel 125. According to another embodiment, the drive wheel 125 is toothed, and the wheel rail 115 is toothed as well. This embodiment allows strong attachment of the drive wheel 125 with the wheel rail 115, and prevents slipping of the drive wheel 125 from the wheel rail 115. It should be noted that the embodiment of toothed drive wheel 125 and a corresponding toothed wheel rail 115 is only exemplary and should not be considered a limiting the scope of the present subject matter. Any type, structure and mechanism of attachment of the drive wheel 125 with the wheel rail 115 is under the scope of the present subject matter.

Also seen in FIGS. 4A-B, is the cutting tool 300 attached to the vehicle 120. The shovel rail 113 shown in FIG. 3A is present also in the shovel 402 shown in FIGS. 4A-B, and is configured to support the cutting tool 300 during its movement together with the vehicle 120. This is achieved, according to one embodiment, by a protrusion 129 that protrudes from the vehicle 120, or from the cutting tool 300, towards the shovel rail 113. According to another embodiment, the protrusion 129 is configured to engage with the shovel rail 113 and slide along the shovel rail 113. This embodiment stabilizes the tool 300 during the movement of the vehicle 120 along the width of the shovel 402.

Still referring to FIGS. 4A-B, according to one embodiment, the shovel rail 113 has a low friction coefficient. In other words, the shovel rail 113 is smooth, thus allowing sliding of the vehicle 120, or the tool, or the protrusion 129 to slide along the shovel rail. Any mechanism for rendering the shovel rail 113 as having a low friction coefficient, or being smooth, is under the scope of the present subject matter. For example, the surface of the shovel rail 113 is made of a material having a low friction coefficient. An advantage of this embodiment is that the smooth shovel rail 113 allows rapid sliding of the vehicle 120, or the tool, or the protrusion 129, along the shovel rail 113, without needing to invest high energy in moving the vehicle 120, or the tool.

According to another embodiment, the shovel rail 113 has a high friction coefficient. In other words, the shovel rail 113 is rough, thus stabilizing the vehicle 120, or the tool, or the protrusion 129 during its movement along the shovel rail 113. Any mechanism for rendering the shovel rail 113 as having a high friction coefficient, or being rough, in under the scope of the present subject matter. For example, the surface of the shovel rail 113 is made of a material having a high friction coefficient. In another example, the surface of the shovel rail 113 comprises bumps, or bulges, or teeth that prevent smooth sliding of the vehicle 120, or the tool, or the protrusion 129 along the shovel rail 113. An advantage of this embodiment is that the rough shovel rail 113 stabilizes the vehicle 120, or the tool during its movement along the shovel rail 113.

Another embodiment shown in FIGS. 4A-B relates to the transmission of electrical power to the vehicle 120 and the tool, for example the cutting tool 300, and transmission of date to and from the tool, or an imaging device and the like, through the endoscope 60. According to one embodiment, transmission of electrical power through the endoscope 60 to components that function on the shovel 402, or at the vicinity of the shovel 402, is performed by at least one cable 652, for example an electricity cable 652. According to another embodiment, transmission of data to, or from, or to and from, components that function on the shovel 402, or at the vicinity of the shovel 402, is performed by at least one cable 652, for example a data transmission cable 652. According to still another embodiment, a plurality of cables 652 is bundled in a cable braid 65, as can be seen in FIGS. 4A-B.

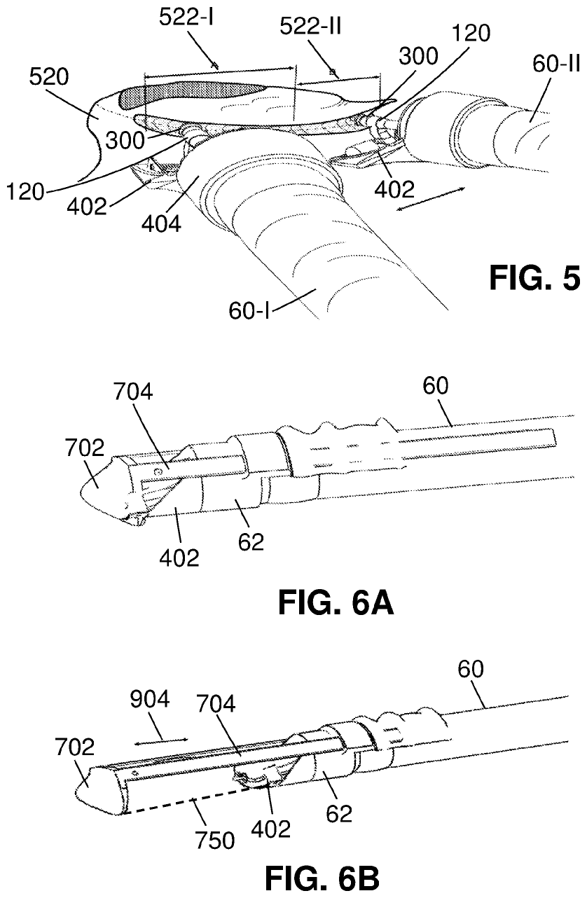

Referring now to FIG. 5, schematically illustrating, according to an exemplary embodiment, two positions of an endoscope comprising a shovel dissecting a target tissue. FIG. 5 shows an endoscope 60 comprising a shovel 402 in a first position 60-I in a vicinity of a target tissue 520. In this position, a cutting tool 300, carried by a vehicle 120 that protrudes from the endoscope 60, cuts a first incision 522-I at a basis of the target tissue 520. Since the endoscope 60 comprises a shovel 402, the vehicle 120 moves side to side along a width of the shovel 402, for example in one of the mechanisms previously described and shown in FIGS. 3A-B and 4A-B. As a result, the first incision 522-I at the basis of the target tissue 520 has a length designated with the line 522-I. The length of the first incision 522-I is larger than an incision made by a prior art endoscope 60, lacking a shovel 402, because the movement of a vehicle 120 carried by the prior art endoscope 60 is limited, compared to the vehicle 120 carried by the endoscope 60 comprising a shovel 402. This embodiment shows one of the advantages of the endoscope 60 comprising a shovel 402 of the present subject matter, compared to prior art endoscopes 60 lacking a shovel 402.

After the first incision 522-I is made, the endoscope 60 moves aside to a second position 60-II, and the cutting tool 300 makes a second incision 522-II at the basis of the target tissue 520, which is a continuation of the first incision 522-II. In this manner it is ultimately possible to cut the entire target tissue 520 and remove the target tissue 520.

FIG. 5 shows a virtual border 523 between the first incision 522-I and the second incision 522-II. The border 523 designates the place on the target tissue 520 where the first incision 522-I ends and the second incision 522-II starts. In this embodiment, the second incision 522-II starts where the first incision 522-I starts. Therefore, the length of the second incision 522-II can be substantially equal to the length of the first incision 522-I. This can be achieved for example by making the first incision 522-I when the endoscope 60 is in the first position 60-I. Then moving the endoscope 60 to the second position 60-II and starting to cut the second incision 522-II substantially at the point where the first incision 522-I ends.

However, according to another embodiment, there can be an overlap between the first incision 522-I and the second incision 522-II. This can be achieved for example by making the first incision 522-I when the endoscope 60 is in the first position 60-I. Then moving the endoscope 60 to the second position 60-II and starting to cut the second incision 522-II at a point within the first incision 522-I.

It should be noted that the embodiment shown in FIG. 5 relates to cutting a target tissue 520 only as an example. This embodiment can relate to any manipulation of a target tissue 520 that requires movement of a tool along a width of the shovel 402, for example injection of a substance into the target tissue, imaging of specific locations on the target tissue 520 and the like.

Referring now to FIGS. 6A-B, schematically illustrating, according to an exemplary embodiment, a perspective view of a collapsed state and an extended state, respectively, of a gripper attached to an endoscope comprising a shovel. FIGS. 6A-B show an endoscope 60 comprising a shovel 402, and additionally comprising a gripper 702. Thus, according to one embodiment, the endoscope 60 comprising a shovel 402 further comprising a gripper 702 attached to a distal edge 62 of the endoscope 60. The gripper 702 is configured to grip a target tissue 520 in a vicinity of the distal edge 62 of the endoscope 60, or more particularly, in a vicinity of the shovel 402 of the endoscope 60. According to another embodiment, the gripper 702 is attached to the distal edge 62 with an arm 704. According to yet another embodiment, the arm 702 is fixed and extends in a manner that forms a distance 750 between the gripper 702 and the distal edge 62 of the endoscope 60, or between the gripper 702 and the shovel 402 that is attached to the distal edge 62 of the endoscope 60, as shown in FIG. 5B. The gripper 702 can have any size and shape that enables the gripper 702 to grip the target tissue 502. When the gripper 702 is used, the target tissue 502 is gripped between the gripper 702 and the distal edge 62, or the shovel 402, at the distance 750 in-between.

According to a further embodiment, the arm 704 is extendable. Thus, the arm 704 can be in two states—a collapsed state and an extended state. The arm 704 in the collapsed state is shown in FIG. 6A. The collapsed state of the arm 704 is used, for example, when the endoscope 60 is transferred through the patient's body toward a target site, for example a target tissue 520 that is to be treated. In the collapsed state of the arm 702, shown in FIG. 6A, the gripper 704 is close to the distal edge 62 of the endoscope 60, or to the shovel 402 that is attached to the distal edge 62 of the endoscope 60.

The arm 704 in the extended state is shown in FIG. 6B. The arm 702 is extended, for example when there is a need to grip a target tissue 520, for example during cutting of the target tissue 520. Arrow 904 shows the possible movement direction of the extendable arm 704 and the gripper 702 attached to the extendable arm 704, namely the extendable arm 704 and the gripper 702 attached to the extendable arm 704 move in a direction which is a continuation of the length of the endoscope 60, away from the distal edge 62 of the endoscope 60, or away from the shovel 402, or towards the distal edge 62 of the endoscope, or towards the shovel 402.

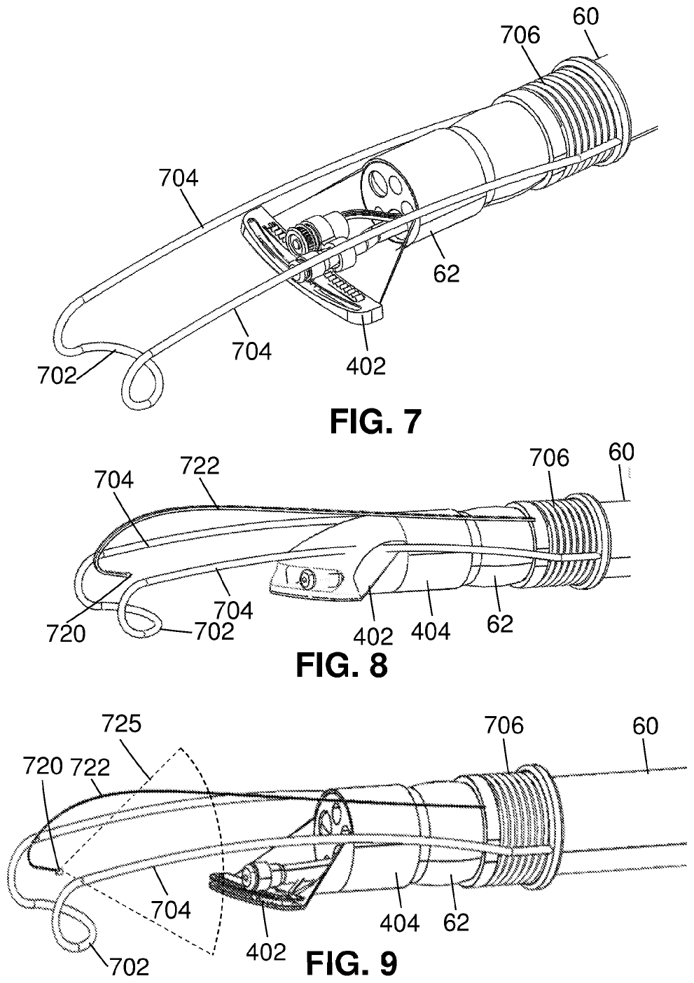

Referring now to FIG. 7, schematically illustrating, according to an exemplary embodiment, another structural embodiment of a gripper and an arm. The gripper 702 and arm 704 shown in FIG. 7 are similar in function to the embodiments described in relation to FIGS. 6A-B. However, the gripper 702 and arm 704 shown in FIG. 7 are different in structure, in that the gripper 702 and arm 704 are made of one piece, for example a wire that is bent. An elongated part of the wire forms the arm 704 and the bent part of the wire forms the gripper 702.

According to another embodiment, the arm 704 is configured to attached to the endoscope 60. According to yet another embodiment, the arm 704 further comprises an arm connector 706 configured to connect the arm 704 to the endoscope 60. Any type of arm connector 706 is under the scope of the present subject matter. An exemplary embodiment of the arm connector 706 is shown in FIG. 7. According to this embodiment, the arm connector 706 is attached to the arm 704 and is configured to embrace the endoscope 60 in a manner that connects the arm 704 to the endoscope 60. According to one exemplary embodiment, the arm connector 706 has a structure of a sleeve that is configured to embrace the endoscope 60 and provide axial movement to the gripper 702 arm out of the scope distal tip transport position to duck on the far side of the elevated target tissue. According to another embodiment, the arm connector 706 has a structure of a coil, as seen in FIG. 7A, that is configured to embrace the endoscope 60. According to yet another embodiment, the connector 706 has a structure of a flexible stent tube.

According to one embodiment, the arm connector 706 has a shape memory that tends to pull the arm 704 and the gripper 702 towards the endoscope 60. According to another embodiment, the arm 704 and gripper are configured to be pushed away from the endoscope 60 by an operator of the endoscope 60. Any mechanism can be used to push the arm 704 and gripper 702 away from the endoscope 60, for example by manually pushing with a cable, or by at least one motor. These embodiments allow easy grasping of a target tissue 520 by the gripper 702. For example, when the endoscope 60 reaches a target tissue 520, the arm 704 and gripper 702 are pushed away from the endoscope 60 in a manner that brings the gripper 702 to a rear side of the target tissue 520 compared to the endoscope 60, when the target tissue 520 is positioned between the shovel 402 and the gripper, while the arm 704 is positioned above the target tissue 520. Then, the pushing of the arm 704 and gripper 702 is released, and as a result the arm 704 and gripper move towards the endoscope 60, because of the shape memory of the arm connector 706 mentioned above. This results in gripping of the target tissue 520 by the gripper 702, and elevation of the target tissue 520 by the gripper 702, for example when such elevation of the target tissue 520 is desired.

Referring now to FIG. 8, schematically illustrating, according to an exemplary embodiment, an endoscope comprising a shovel, a gripper and an arm further comprising a rear imaging device. According to the embodiment shown in FIG. 8, the endoscope 60 comprising a shovel 402, and gripper 702 and arm 704, further comprises a rear imaging device 720 configured to acquire images from an opposite side of the target tissue 520 that is gripped by the gripper 702. The rear imaging device 720 is positioned near the gripper 702 and is connected to the arm connector 706, or directly to the endoscope 60, with an imaging device arm 722. According to one embodiment, the imaging device arm 722 is configured to connect the rear imaging device 720 to the endoscope 60. According to another embodiment, the imaging device arm 722 is configured to connect the rear imaging device 720 to the arm connector 706. However, according to yet another embodiment, the rear imaging device 720 is attached to the gripper 702. According to a further embodiment, the imaging device arm 722 comprises wires that are configured to transmit signals, through the endoscope, to an input device positioned out of the patient's body and configured to acquire signals from the rear imaging device 720 and process the signals. However, according to an additional embodiment, the rear imaging device 720 is wirelessly connected to the input device and therefore, the imaging device arm 722 may not comprise wires.

According to one embodiment, the rear imaging device 720 is configured to be moved by an operator of the endoscope 60. Any type of movement of the rear imaging device 720 and mechanism for moving the rear imaging device 720, and controlling the movement of the rear imaging device 720, are under the scope of the present subject matter. For example, the rear imaging device 720 is configured to swivel side-to-side, or upwards and downwards and the like. Movement of the rear imaging device 720 can be achieved mechanically, for example by using cables, or with at least one motor, and the like.

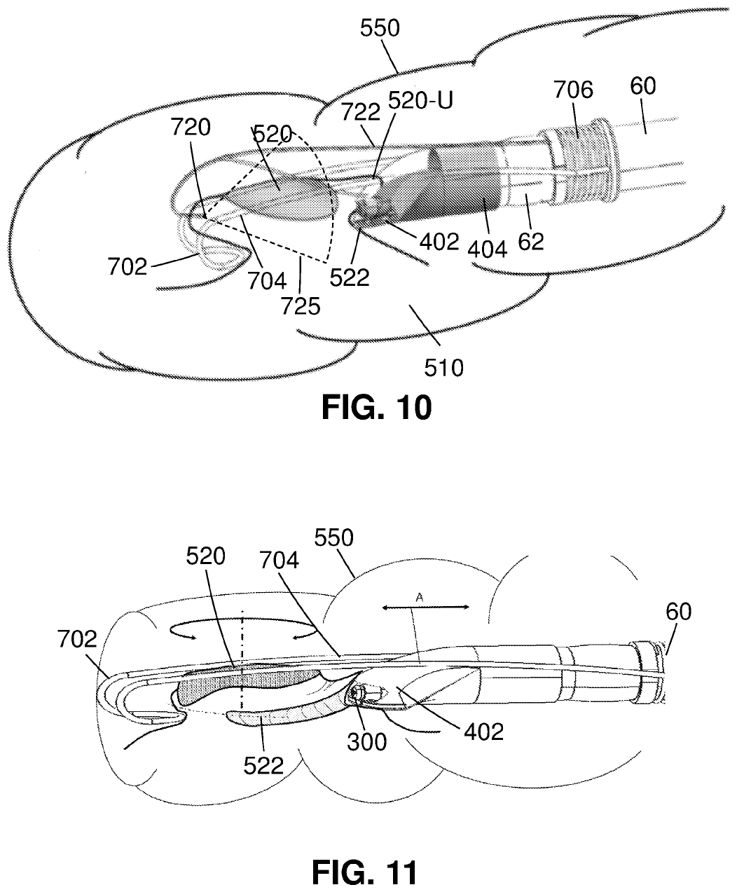

Referring now to FIG. 9, schematically illustrating, according to an exemplary embodiment, a field of vision of a rear imaging device. The endoscope 60 comprising a shovel 402, a gripper 702, an arm 704, a rear imaging device 702 and an imaging device arm 722 shown in FIG. 9 is essentially similar to the same shown in FIG. 8. FIG. 9 additionally shows a field of vision 725 of the rear imaging device 720. The field of vision 725 has a three-dimensional funnel-like shape that extend from the rear imaging device 720. Any object that falls within the field of vision 725 is imaged by the rear imaging device 720. According to the embodiment shown in FIGS. 8 and 9, the rear imaging device 720 points toward the endoscope 60. Therefore, any object that is positioned between the rear imaging device 720 and the endoscope 60, or more particularly, between the rear imaging device 702 and the shovel 702, and falls in the field of vision 725 of the rear imaging device 720, can be imaged. An advantage of this embodiment is illustrated in the following FIG. 10.

Referring now to FIG. 10, schematically illustrating, according to an exemplary embodiment, a side view of an endoscope comprising a shovel imaging a rear view of a target tissue gripped with a gripper inside a cavity. As can be seen in FIG. 10, an endoscope 60 comprising a shovel 402 is inserted into a cavity 550, for example an intestine, in a patient's body, in order to image a target tissue 520 from a rear view. The endoscope 60 further comprises a gripper 702 and s rear imaging device 720 as shown in FIGS. 8-9. A prior art endoscope 60 comprises a prior art imaging device that is attached to a front side of the endoscope 60 and configured to image a view that is in front of the endoscope 60. Thus, if a prior art endoscope 60 would have been used in the scenario shown in FIG. 10, the prior art imaging device of the prior art endoscope 60 would have been imaged only a part of the target tissue 520 that faces the endoscope 60. By using the prior art imaging device of the prior art endoscope 60 it is impossible to image a side of the target tissue 520 that is in an opposite side, compared to the endoscope 60, because it is hidden from the prior art imaging device of the prior art endoscope 60.

However, as can be seen in FIG. 10, the rear imaging device 720 is pointing towards the endoscope 60, and when the rear imaging device 720 is positioned in front of a rear side of the target tissue 520, as seen in FIG. 10, the rear side of the target tissue 520 is within the field of vision 725 of the rear imaging device 720, thus enabling imaging of the rear side of the target tissue 520.

According to one embodiment, in the scenario shown in FIG. 10, the gripper 702 can be used for gripping the target tissue 520 at the opposite side of the target tissue 520, and even elevate the target tissue 520, thus facilitating the imaging of the opposite side of the target tissue 520.

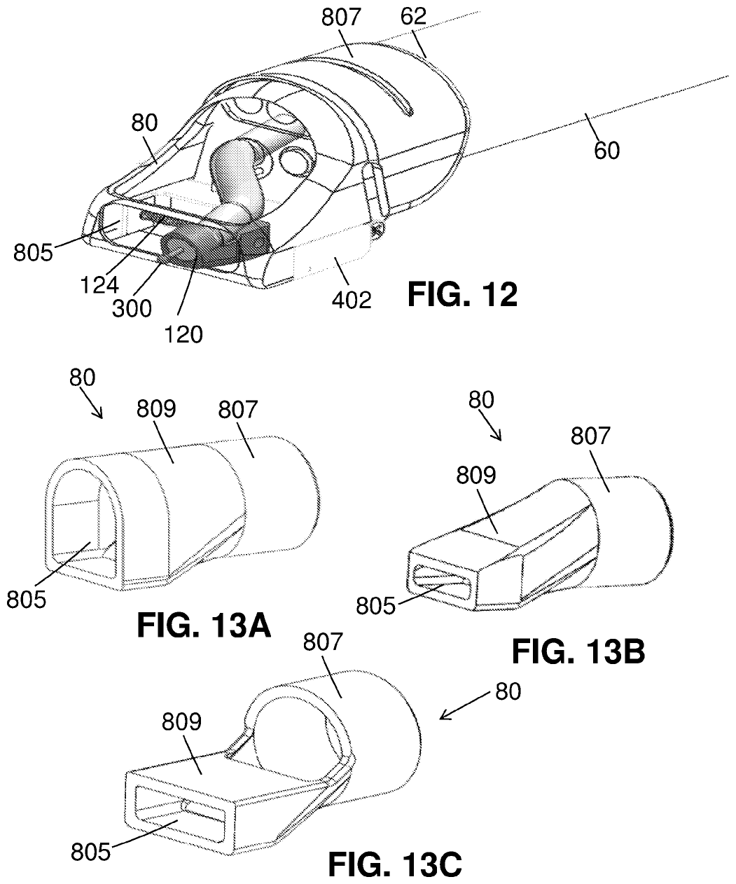

Another advantageous feature of the shovel 402 that is shown in FIG. 10 relates to a depth of an incision 522 that is made in a target tissue 520. When using a prior art endoscope 60, the depth of the incision 522 is limited to the length of protruding part of the cutting tool 300. This is because the width of the endoscope 60 is large, and the endoscope cannot penetrate into the incision 522 made in the target tissue 500. However, the height of the shovel 402 is smaller than the height of the endoscope 60, thus enabling penetration of the shovel 402 into the incision 522 and thereby increasing the depth of the incision 522 in the target tissue 520. As can be seen in FIG. 10, the shovel 402 penetrates the incision 522, and as a result a part 520-U of the target tissue 520 lies over the shovel 402. This situation can interfere with the function of the endoscope 60, for example musk the vision field of the endoscope's 60 imaging device, or expose components on the shovel 402, like the vehicle 120, the tool, and the like, to direct contact with tissue and moisture. A solution for this situation is described, for example, in FIGS. 12-13, hereinafter.

Referring now to FIG. 11, schematically illustrating, according to an exemplary embodiment, a side view of an endoscope comprising a shovel and a gripper dissecting a target tissue gripped with a gripper. As can be seen in FIG. 11, an endoscope 60 comprising a shovel 402 is inserted into a cavity 550, for example an intestine, in a patient's body, in order to cut a target tissue 520. The endoscope 60 further comprises a gripper 702 similar to the gripper 702 shown previously. A cutting tool 300 is positioned on the shovel 402, according to embodiments described above. When the cutting tool 300 dissects the target tissue 520, the gripper 702 grips the target tissue 520 at an opposite side relative to the cutting tool 300. This is achieved due to the arm 704 that holds the gripper 702 at a distance from the shovel 702. Thus, the gripper 702 stabilizes the target tissue 520 and allows easier and more accurate dissection of the target tissue 520. As a result, the incision 522 in the target tissue 520 can be made with high accuracy, in terms of position of the incision 522 in the target tissue 520 and width and length of the incision 522. This is another advantage of using the gripper 702 in combination with the shovel 402, because when a target tissue 520 is dissected with a prior art endoscope 60 comprising a cutting tool 300, there is no grasping and stabilization of the target tissue 522, and since the target tissue 520 is [symbol] in nature, there is a limit in the accuracy of the position, width and length of the incision 522.

Referring now to FIG. 12, schematically illustrating, according to an exemplary embodiment, an endoscope comprising a shovel and a shovel cover. However, before describing the various embodiments of the shovel cover 80, FIG. 12 illustrates the embodiment of the second mechanism for facilitating movement of a vehicle 120 along a width of the shovel 402, shown in FIG. 3B. FIG. 12 shows the screw 124 positioned along a width of the shovel 402. However, FIG. 12 does not show the bolt 126 that is attached to the vehicle 120 and screwed over the screw 124, and the motor 122 positioned substantially near a side of the shovel 402. wherein the screw 124 is attached to the motor 122 and the motor 122 is configured to rotate the screw 124, as described in detail in the description of FIG. 3B above.

According to one embodiment, shown in FIG. 12, the endoscope 60 comprising a shovel 402 further comprises a shovel cover 80 configured to cover the shovel 402 from above and allow protrusion of a tool 300 out from the shovel 402. As can be seen in FIG. 12, the shovel cover 80 covers the shovel 402 and a vehicle 120 and a tool in a form of a cutting tool 300 that are on the shovel 402. In addition, the shovel cover 80 comprises an opening 805 at a front side relative to the distal edge 62 of the endoscope. The opening 805 is configured to allow protrusion of a tool from the shovel 402. Thus, the cutting tool 300 shown in FIG. 12 can dissect a target tissue 250 by protruding through the opening 805 of the shovel cover 80.

Another embodiment of the shovel cover 80 is that the shovel cover is configured to protect the shovel 402 and components that are on the shovel 402, for example a vehicle 120, a tool, and the like, from a physical damage that can occur during the insertion of the endoscope 60 into the patient's body, and from moisture that may harm the components, like blood, mucus and the like.

According to yet another embodiment, the shovel cover 80 is substantially transparent. Therefore, the vehicle 120 is partially seen through the shovel cover 80, as shown in FIG. 12. This embodiment of the transparent shovel cover 80 allows imaging with a prior art imaging device positioned at a distal edge of the endoscope 60, for example aside the vehicle 120. Therefore, there is no need to remove, or open, the shovel cover 80, or protrude the imaging device through the opening 805 of the shovel cover 80, in order to obtain images with the prior art imaging device of the endoscope 60.

Any mechanism for attaching the shovel cover 80 over the shovel 402 is under the scope of the present subject matter. According to one embodiment, the shovel cover 80 permanently covers the shovel 402. According to another embodiment, the shovel covert 80 is removable. According to an exemplary embodiment, shown in FIG. 12, a cover connector 807 is attached to the shovel cover 80 and is configured to connect the shovel cover 80 to the endoscope 60, or to the distal edge 62 of the endoscope 60. For example, the cover connector 807 is in a form of a sleeve that engages with the distal edge 62 of the endoscope 60, and connects with the distal edge 62, either permanently, or removably.

Any size and shape of the shovel cover 80 is under the scope of the present subject matter. For example, the shovel cover 80, shown in FIG. 12, is inclined from the distal edge 62 toward the opening 805, and concave. This exemplary embodiment of the shape of the shovel cover 80 allows sliding of moisture and dirt off the shovel cover 80, for example to allow vision through a transparent shovel cover 80 with a prior art imaging device from the endoscope 60, despite an accumulation of moisture and dirt on the shovel cover 80, because they slide off the shovel cover 80. Additional embodiments of the size and shape of the shovel cover 80 are shown in FIG. 13A-C.

Referring now to FIGS. 13A-C, schematically illustrating, according to an exemplary embodiment, various embodiments of size and shape of a shovel cover. All the shovel covers 80 shown in FIGS. 13A-C comprise a cover connector 807. Since the cover connector 807 is configured to engage with a distal edge 62 of the endoscope 60, or with the endoscope 60, the size and shape of the shovel connector 807 is substantially similar in all the types of the shovel cover 80 shown in FIGS. 13A-C. However, it should be noted that the shape and size of the cover connector 807 can be different, for example in order to adapt to sizes and shapes of various types of endoscopes 60, or distal edges 62 of endoscopes 60.

According to another embodiment, shown in FIGS. 13A-C, the shovel cover comprises also a covering element 809. Thus, according to this embodiment, the shovel cover 80 comprises a covering element 809 configured to cover the shovel 402, and a cover connector 807 attached to the covering element 809 and configured to connect the shovel 80 to the endoscope 60, or to the distal edge 62 of the endoscope 60. According to embodiments mentioned above, the covering element 809 is substantially transparent, according to another embodiment, the covering element 809 is opaque, in other words, not transparent.

Any size and shape of the covering element 809 is under the scope of the present subject matter. It should be noted that the types of covering element 809 shown in FIGS. 13A-C are only exemplary and should not be considered as limiting the scope of the present subject matter. For example, according to one embodiment, shown in FIG. 13A, the covering element is substantially cylindrical and has a diameter substantially similar in size to a diameter of the endoscope 60. According to another embodiment, shown in FIG. 13B, the covering element 809 is substantially inclined, having at an edge adjacent to the endoscope 60 a height similar to a height of the endoscope 60, and at an edge distal from the endoscope 60 a height lower than the height of the endoscope 60. According to a further embodiment, shown in FIG. 13C, the covering element 809 is substantially rectangular. According to yet a further embodiment, the height of the rectangular covering element 809 is lower than the height of the endoscope 60.

Another embodiment that is similar in all the shovel covers 80 shown in FIGS. 13A-C. is that the shovel cover 80 comprises an opening 805 at a distal edge from the endoscope 60. As mentioned above, the opening 805 is configured to allow protrusion of, for example, a tool from the shovel 402, as shown in FIG. 12. According to another embodiment, the opening 805 allows imaging with a prior art imaging device of the endoscope 60. The opening 8 can have any size and shape. For example, the shovel cover 13A shown in FIG. 13A comprises a large opening 805, having a semi-cylindrical shape, with a diameter substantially similar to a diameter of the endoscope 60. In contrast, the shovels 80 shown in FIGS. 13B-C comprise smaller openings 805, having a rectangular shape, and having a height smaller than a height of the endoscope 60. The size and shape of the opening 805 can be adapted for example to the type, size and shape of the tool that is used.

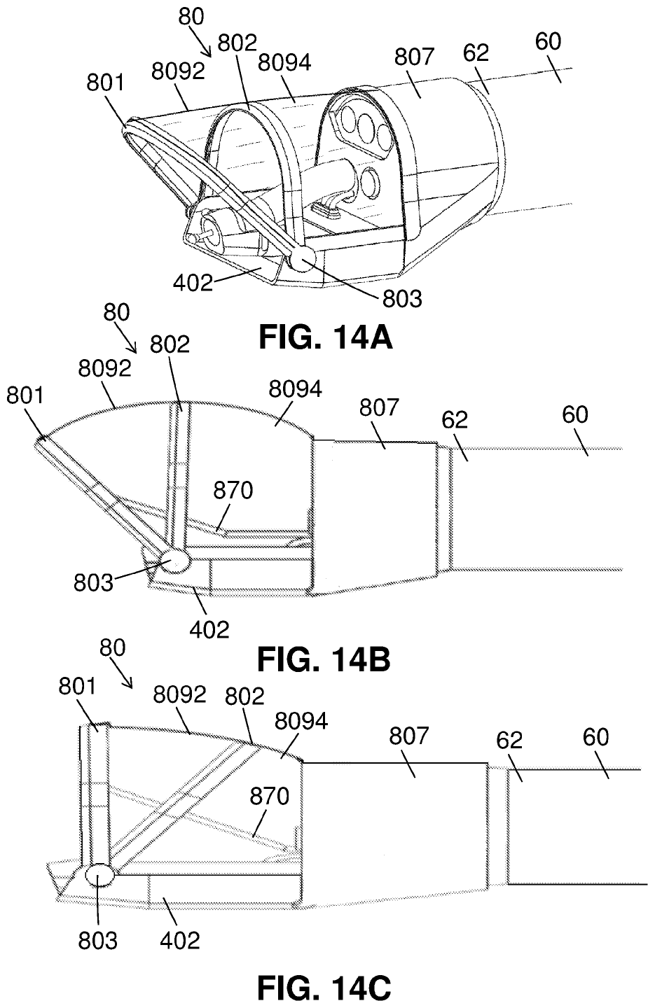

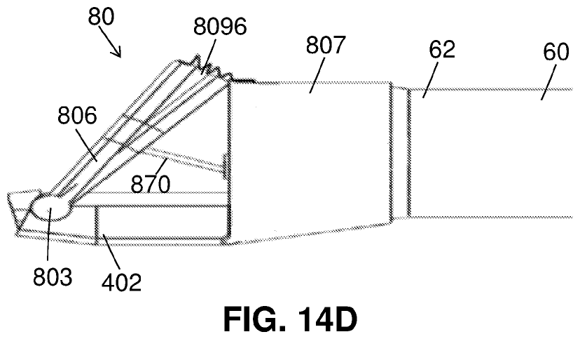

Referring now to FIG. 14A, schematically illustrating, according to an exemplary embodiment, a side perspective view of a foldable shovel cover in a spread state. According to one embodiment, the covering element 809 of the shovel cover 80 is at least partially foldable. According to this embodiment, shown in FIG. 14A, the covering clement 809 of the shovel cover 80 comprises a first cover 8092 and a second cover 8094. The first cover 8092 is spread between a first frame 801 and a second frame 802, and the second cover 8094 is spread between the second frame 802 and the cover connector 807. According to one embodiment, the first frame 801 and the second frame 802 are axially attached to the shovel 402 with a cover axis 803. The cover axis 803 can be placed at any position on the shovel 402. According to one embodiment, shown in FIG. 14A, the cover axis 803 is placed at a distal edge of the shovel 402. According to one embodiment, the first frame 801 is configured to swivel about the cover axis 803. According to another embodiment, the second frame 802 is configured to swivel about the cover axis 803. According to yet another embodiment, the first frame 801 and the second frame 802 are configured to swivel about the cover axis 803.

Referring now to FIGS. 14B-C, schematically illustrating, according to an exemplary embodiment, a side view of a foldable shovel cover in a spread state and a folded state, respectively. In FIG. 14B, the foldable shovel cover 80 is in a spread state, similarly to the foldable shovel cover shown in FIG. 14A. The first frame 801 is swiveled forward, away from the endoscope 62, thus causing the first cover 8092 to cover a front portion of the shovel 402. In this state, the second frame is positioned substantially vertically in relation to the shovel 402, and the second cover 8094 covers a central and rear part of the shovel 402.

According to one embodiment, the foldable shovel cover 80 in the spread state, as shown in FIGS. 14A-B, is configured to allow a smooth and easy movement of the endoscope inside the cavity 550. This is achieved, for example, by the shape of the foldable shovel cover 80 seen in FIGS. 14B, particularly the shape of the first cover 8092 and the second cover 8094. According to another embodiment, the foldable shovel cover 80 in the spread state has a curved shape, resembling a hydrodynamic shape, that allows smooth movement of a front size of the endoscope 60 through tissues, and through cavities 550 in particular.

In FIG. 14C, showing a foldable shover cover 80 in a folded state, both the first frame 801 and the second frame 802 are swiveled backwards, towards the endoscope 60. As a result, the first cover 8092 covers the central part of the shovel 402, and the second cover 8094 covers the rear side of the shovel, while the front part of the shovel 402 is not covered.

FIGS. 14B-C additionally show a frame arm 870 configured to be actuated by an operator of the endoscope 60 and push the first frame 801 forward, or backwards, as desired. According to one embodiment, one end of the frame arm 870 is attached to the first frame 801, and another side of the frame arm 870 is connected, either directly, or with wires, or wirelessly to a control panel, or a handle, operated by the operator of the endoscope 60. Pushing the frame handle 870 forward moves the first frame 801 forward, while pulling the frame handle 870 backward moves the first frame backward.

It should be noted that the embodiment of the frame arm 870 connected to the first arm 801, shown in FIGS. 14B-C, is only exemplary and should not be considered as limiting the scope of the present application. According to another embodiment, the frame arm 870 is connected to the second frame 8094, and is configured to push forward, or pull backwards, the second frame 802.

According to one embodiment, the first cover 8092 is made of a folding material, for example a folding sheet of nylon, or a fabric, and the like. According to another embodiment, the first cover 8092 is rigid.

According to one embodiment, the second cover 8094 is made of a folding, for example a folding sheet of nylon, or a fabric, and the like. According to another embodiment, the second cover 8094 is rigid.

According to the embodiment of the first cover 8092 made of a folding material, the first cover 8092 is spread out as the first frame 801 swivels away from the endoscope 60, as shown in FIGS. 14A-B. According to the embodiment of the first cover 8092 made of a rigid material, the first cover 8092 is pooled out as the first frame 801 swivels away from the endoscope 60, as shown in FIGS. 14A-B.

According to the embodiment of the second cover 8094 made of a folding material, the second cover 8094 is spread out as the second frame 802 swivels away from the endoscope, as shown in FIGS. 14A-B. According to the embodiment of the second cover 8094 made of a rigid material, the second cover 8094 is pooled out as the second frame 802 swivels away from the endoscope, as shown in FIGS. 14A-B.

According to one embodiment, in the spread state shown in FIG. 14C, according to the embodiment of the second cover 8094 made of a folding material, the second cover 8094 folds, or converges, as the second frame 802 swivels towards the endoscope 60. According to the embodiment of the second cover 8094 is pushed inside the endoscope, or under the cover connector 807 as the second frame 802 swivels towards the endoscope 60.

Referring now to FIG. 14D, schematically illustrating, according to an exemplary embodiment, a side view of a folding shovel cover. According to the embodiment shown in FIG. 14D, the shovel cover 80 comprises a cover frame 806 axially attached to the shovel 402 with a cover axis 803, and a folding cover 8096, one side of the folding cover 8096 attached to the cover frame 806, and another side of the folding cover 8096 attached to the cover connector 807. When the cover frame 806 swivels towards the endoscope 60, as shown in FIG. 4D, the folding cover 8096 folds, and the shovel 402 is not covered, namely exposed. When the cover frame 806 swivels away from the endoscope 60, the folding cover 8096 spreads, and the shovel 402 is covered. According to another embodiment shown in FIG. 14D, the shovel cover 80 further comprises a frame arm 870. Embodiments of the frame arm 870 are described above, in relation to FIGS. 14B-C. According to an additional embodiment, the folding cover 8096 shown in FIG. 14D is attached to two frames, similarly to the shovel cover 80 shown in FIGS. 14A-C.

According to one embodiment, the foldable shovel cover 80, including any embodiment of the foldable shovel cover 80 described above, is configured to manipulate the target tissue 520. Any type of manipulation of the target tissue 520 that can be performed by the foldable shovel cover 80 is under the scope of the present subject matter. According to one embodiment, the foldable shovel cover 80 is configured to elevate the target tissue 520. According to another embodiment, the foldable shovel cover 80 is configured to push the target tissue 520. According to yet another embodiment, the foldable shovel cover 80 is configured to perform any manipulation of the target tissue 520 that allows visualization of the target tissue 520 with imaging devices. According to still another embodiment, the foldable shovel cover 80 is configured to perform any manipulation of the target tissue 520 that allows accurate dissection of the target tissue 520, for example with a cutting tool 300. In general, the foldable shovel cover 80 is configured to assist in controlling the position and condition of the target tissue 520 during manipulation of the target tissue 520. According to an additional embodiment, the first frame 801 of the foldable shovel cover 80 is configured to perform the aforementioned embodiments that relate to the controlling the position and condition of the target tissue 520.

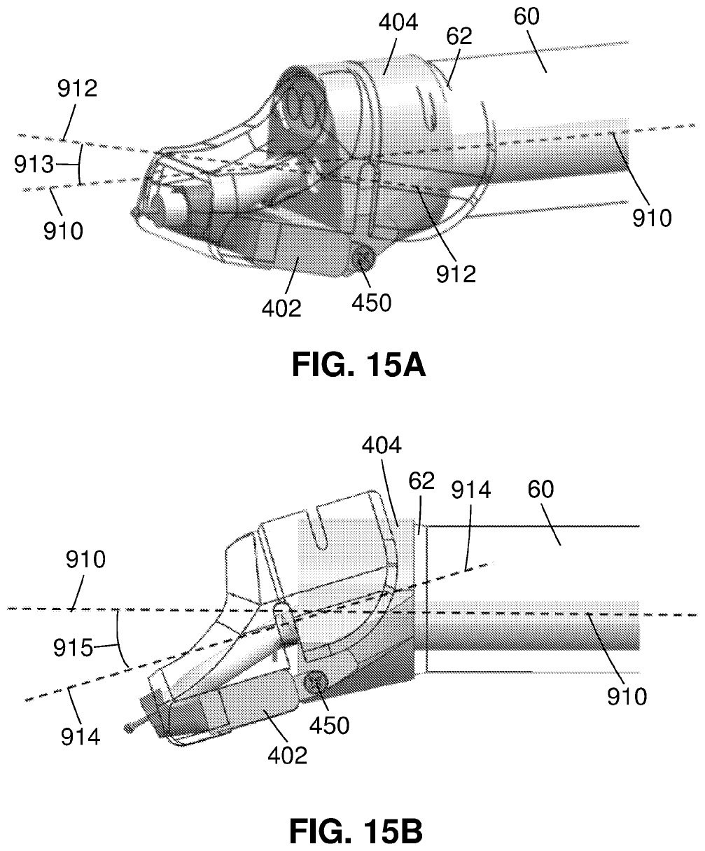

Referring now to FIGS. 15A-B, schematically illustrating, according to an exemplary embodiment, a side perspective view of a shovel swiveling upwards and a shovel swiveling downwards, respectively. The former drawings illustrate a shovel 402 being in line with a length of the endoscope 60. In FIGS. 15A-B, dashed line 910 designates a line of length of the endoscope 60. According to one embodiment, shown in FIGS. 15A-B, the shovel 402 is axially attached to the connector 404 with a horizontal axis 450 positioned at a bottom part of the connector 404. This horizontal axis 450 allows swiveling of the shovel 402 either upwards, or downwards, or upwards and downwards.

FIG. 15A shows a shovel 402 configured to swivel upwards. In FIG. 15A, dashed line 912 designates a line of length of the shovel 402 when the shovel 402 swivels upwards, and angle 913 is the angle that is formed between the endoscope-line-of-length 910 and the swiveling-upwards-shovel-line-of-length 912.

FIG. 15B shows a shovel 402 configured to swivel downwards. In FIG. 15B, dashed line 914 designates a line of length of the shovel 402 when the shovel 402 swivels downwards, and angle 915 is the angle that is formed between the endoscope-line-of-length 910 and the swiveling-downwards-shovel-line-of-length 914.

According to another embodiment, the shovel 402 is configured to swivel upwards, as shown in FIG. 15A, and downwards, as shown in FIG. 15B. The embodiments illustrated in FIGS. 15A-B provide flexibility to the function of the tool, and increase the region of the target tissue 520 that can be manipulated with the tool, without a need to move the endoscope 60.

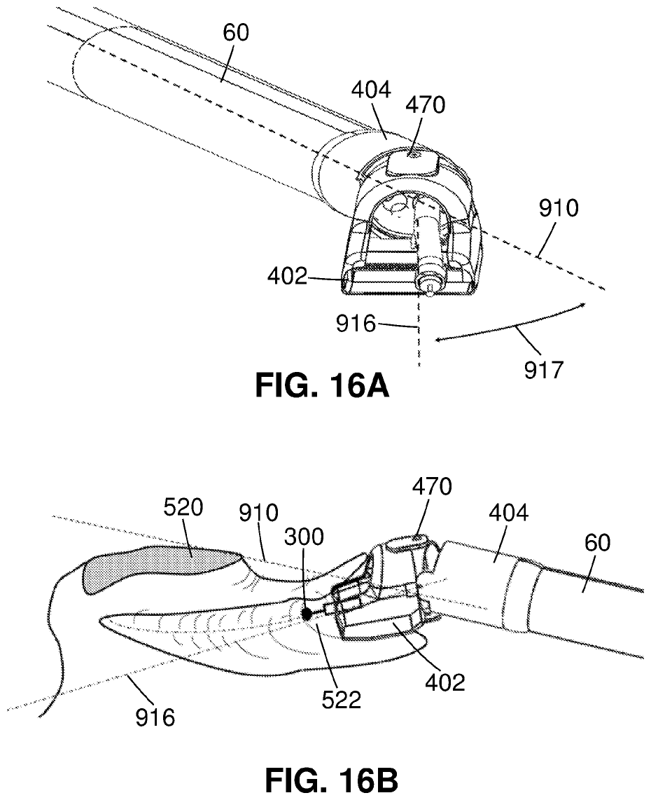

Referring now to FIG. 16A, schematically illustrating, according to an exemplary embodiment, a front perspective view of a shovel configured to swivel side to side. Dashed line 910 designates a line of length of the endoscope 60. According to one embodiment, shown in FIG. 16A, the shovel 402 is axially attached to the connector 404 with a vertical axis 470 positioned at a border between the shovel 402 and the connector 404. This vertical axis 470 allows swiveling of the shovel 402 either to the right, or to the left, or to the right and to the left.

FIG. 16A shows a shovel 402 configured to swivel to the right. In FIG. 16A, dashed line 916 designates a line of length of the shovel 402 when the shovel 402 swivels to the right, and angle 917 is the angle that is formed between the endoscope-line-of-length 910 and the swiveling-to-the-right-shovel-line-of-length 917. Similarly, according to another embodiment, the shovel 402 is configured to swivel to the left, forming an angle between the endoscope-line-of-length 910 and a swiveling-to the-left-shovel-line-of-length. Even though this specific embodiment is not shown, it is easy to visualize the embodiment of the shovel 402 swiveling to the left, based on the shovel 402 swiveling to the right, shown in FIG. 16A. An advantage of the shovel 402 configured to swivel to the right, or to the left, or to the right and the left, is illustrated in FIG. 16B, below

Referring now to FIG. 16B, schematically illustrating, according to an exemplary embodiment, a side perspective view of a shovel configured to swivel to the right, or to the left, or to the right and to the left, dissecting a target tissue. FIG. 16B illustrates a great Advantage of the shovel 402 configured to swivel to the right, or to the left, or to the right and to the left. This embodiment dramatically increases the area that can be influenced by a tool carried by an endoscope 60 comprising a shovel 402 configured to swivel to the right, or to the left, or to the right and to the left. When, for example, the tool is a cutting tool 300, the ability of the shovel 402 to swivel to the sides dramatically increases the size of incision 522 in the target tissue 520 that can be made, compared to endoscopes 60 comprising a fixed shovel 402, as described above, and especially compared to prior art endoscopes 60 that do not comprise a shovel 402. As describes above, the shovel 402 allows movement of a tool, for example a cutting tool 300, for size to size within the width of the shovel 402, without having to move the endoscope 60. This is advantageous over prior art endoscopes 60 that do not comprise a shovel 402, and as a result, the cutting tool 300 is fixed and there is a need to move the entire endoscope 60 from side to side in order to achieve a long and continuous incision 522. Now, the embodiment shown in FIGS. 16A-B further increases the area of influence of the tool, for example dramatically increases the size of the incision 522, without needing to move the endoscope 60.

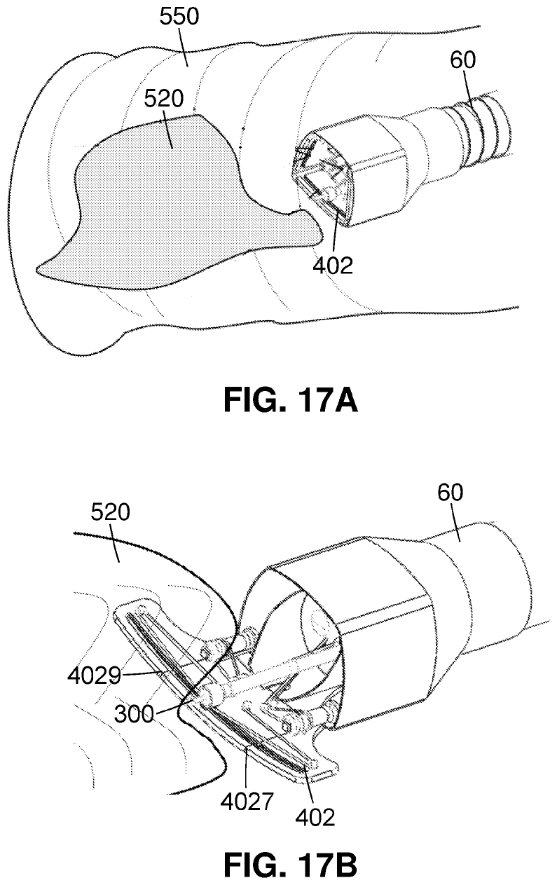

Referring now to FIGS. 17A-B, schematically illustrating, according to an exemplary embodiment, a front perspective view of a foldable shovel in a folded state and an open state, respectively. FIG. 17A shows an endoscope 60 inserted into a cavity 550 and directed towards a target tissue 520. The endoscope 60 comprises a foldable shovel 402 in a folded sate, positioned inside the endoscope 60. The foldable shovel 402 is maintained in the folded state during the transfer of the endoscope in the cavity 550 towards the target tissue 520. When the endoscope 60 approaches the target tissue 520, the foldable shovel 420 is moved out of the endoscope 60 and spreads, or opens, to form the open state shown in FIG. 17B. In the open state, the foldable shovel 420 extends aside beyond a width of an endoscope 60. Any mechanism for folding the foldable shovel 402 is under the scope of the present subject matter. According to an exemplary embodiment, shown in FIG. 17B, the foldable shovel comprises at least one shovel axis. For example, the foldable shovel 402 shown in FIG. 17B comprises two shovel axes-a first shovel axis 4027 and a second shovel axis 4029. According to one embodiment, the foldable shovel 402 is configured to fold at the at least one shovel axis. Thus, as mentioned above, when the endoscope 60 is transferred in the cavity 550 the foldable shovel 402 is folded and maintained inside the endoscope 60, for example in order to prevent contact of the shovel 402 with tissue during the transfer of the endoscope 60. When the endoscope 60 reaches the target tissue 520, the foldable shovel 402 is moved out of the endoscope 60 and opens, for example in order to dissect the target tissue 520, or perform any other manipulation with the folded shovel 402 in the open state. After the manipulation of the target tissue 520 is complete, the foldable shovel 402 folds back to the closed state, and pushed back inside the endoscope 60, as shown in FIG. 17A. Any mechanism for moving the foldable shovel 402 outside and inside the endoscope 60 and for opening the foldable shovel 402 and bringing the foldable shovel back to the closed state is under the scope of the present subject matter.

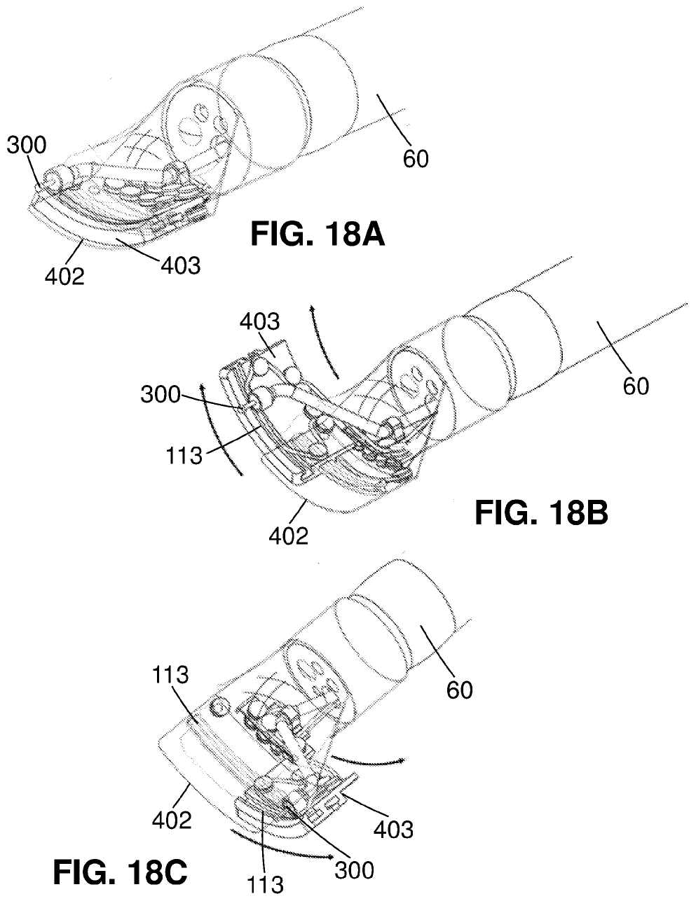

Referring now to FIGS. 18A-C, schematically illustrating, according to an exemplary embodiment, a front perspective view of a shovel comprising a swiveling sub-shovel, in three states. According to one embodiment, shown in FIGS. 18A-C, the shovel 402 further comprises a swiveling sub-shovel 403 positioned over, or under the shovel 402, and configured to swivel aside. An advantage of this embodiment is that it allows extension of the width of the shovel 402, for example beyond the width of the endoscope 60, thereby increasing the area that can be manipulated with a tool, for example a cutting tool 300. FIG. 18A shows the shovel 402 as described above, and the swiveling sub-shovel 403 placed over the shovel 402. In this position. the swiveling sub-shovel 403 does not extend beyond the shovel 402. In FIG. 18B, the swiveling sub-shovel 403 swivels to the right, thus extending beyond the right side of the shovel 402. In FIG. 18C, thew swiveling sub-shovel 403 swivels to the left, thus extending beyond the left side of the shovel 402. According to one embodiment, the swiveling sub-shovel 403 can extend the operational width of the shovel 402 up to threefold, when the width of the swiveling sub-shovel 403 is similar to the width of the shovel 402. In other words, in an embodiment of using for example a cutting tool 300, the swiveling sub-shovel 403 can increase up to threefold the length of an incision 522 made by the cutting tool 300 while the endoscope 60 is fixed in place, when first swiveling the swiveling sub-shovel 403 to the right, for example, and then to the left.

As can be further seen in FIGS. 18B-C, the swiveling sub-shovel 403 is similar to the shove 402. For example, the swiveling sub-shovel 403 comprises a shovel rail 113, like the shovel 402. As a result, a tool, for example a cutting tool 300, can move along the shovel rail 113 of the shovel 402 and on the shovel rail 113 of the swiveling sub-shovel 403, when the swiveling sub-shovel 403 is swiveled aside, either to the right, as shown in FIG. 18B, or to the left, as shown in FIG. 18C.

As mentioned above, according to one embodiment, shown in FIG. 18A, the swiveling sub-shovel 403 is positioned under the shovel 402. According to another embodiment, the swiveling sub-shovel 403 is positioned over the shovel 402. Even though there is no drawing that shows a swiveling sub-shovel 403 positioned over a shovel 402, this embodiment can be easily understood from FIGS. 18A-C.

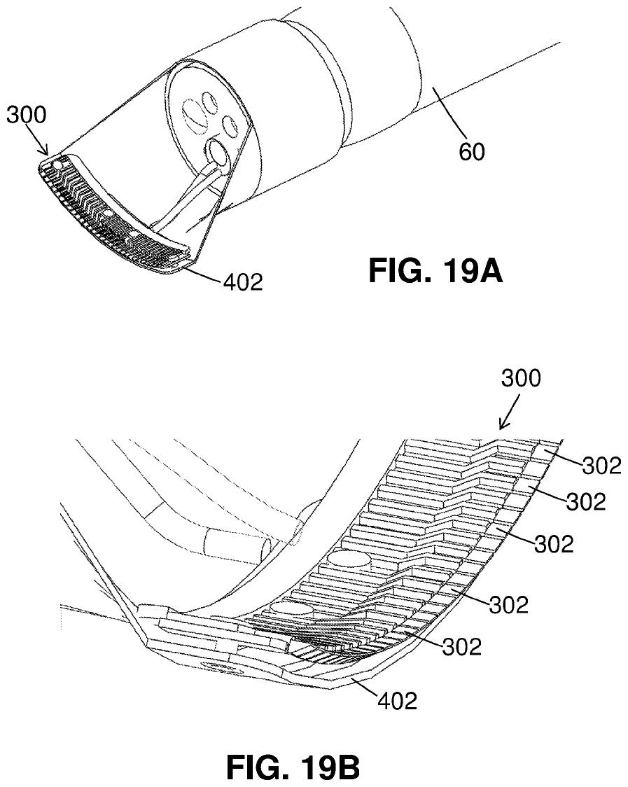

Referring now to FIGS. 19A-B, schematically illustrating, according to an exemplary embodiment, a front perspective view of an endoscope comprising a shovel and a multi-barreled cutting tool, and a side perspective close view of the shovel and the multi-barreled cutting tool, respectively. FIG. 19A shows an endoscope 60 comprising a shovel 402, as described above, and a cutting tool 300 in a form of the multi-barreled cutting tool 300. According to this embodiment, the cutting tool 300 is a radio frequency (RF) cutting tool 300, or a diathermic cutting tool 300. Such cutting tools 300 are configured to dissect a tissue by a radio frequency, or by heating the tissue at high temperatures. Also, the cutting tool 300 shown previously, for example in FIGS. 18A-C, can be a RF cutting tool 300, or a diathermic cutting tool 300. This cutting tool 300 comprises a single barrel that emits the RF, or heats the tissue. As a result, there is a need to move the cutting tool 300 along the shovel 402 in order to dissect the target tissue 520, as described above. However, an advantage of the shovel 402 is that is allows usage of a multi-barreled cutting tool 300. According to this embodiment, the multi- barreled cutting tool 300 comprises a plurality of barrels 302 arranged on the shovel 402. When there is a need to dissect a target tissue at least one barrel 302, positioned for example at a side of the shovel 402, is actuated, for example transmitting a RF, or heat, towards the target tissue 520. Thus, the target tissue 520 is dissected at the area of the actuated at least one barrel 302. Then, in order to continue the dissection of the target tissue 520, the at least one barrel 302 is shut off, and at least one barrel 302 positioned aside the first at least one barrel 302 is actuated, thus dissecting the target tissue 520 at a position aside the first incision. Thus, in this manner, an incision can be made along the width of the shovel 402, by alternately actuating the barrels 302 as described above. This embodiment, of the multi-barreled cutting tool 300 negates mechanical movement of the cutting tool 300, as described previously, thus rendering the dissection of the target tissue 520 more rapid and accurate.

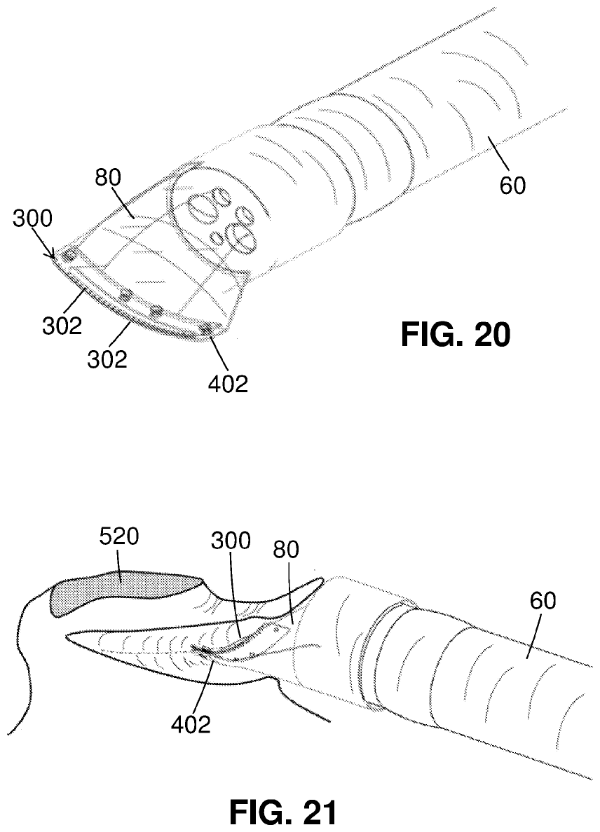

Referring now to FIG. 20, schematically illustrating, according to an exemplary embodiment, a front perspective view of an endoscope comprising a shovel and a multi-barreled cutting tool and a shovel cover. The endoscope 60 shown in FIG. 20, comprises a shovel 402 and a multi-barrel cutting tool, like the embodiment shown in FIGS. 19A-B, as well as a shovel cover 80, as shown for example in FIG. 12. According to one embodiment, the shovel cover 80 is transparent, enabling imaging with a prior art imaging device of the endoscope.

Referring now to FIG. 21, schematically illustrating, according to an exemplary embodiment, a side perspective view of an endoscope comprising a shovel and a multi-barreled cutting tool and a shovel cover, dissecting a target tissue. The dissection of the target tissue 520 with the multi-barreled cutting tool 300 is performed as described previously, in relation to FIGS. 19A-B. During the dissection of the target tissue 520, a part of the target tissue slides over the cover 80, thus on one hand enabling dipper dissection of the target tissue 520, compared to prior art endoscopes 60, and on the other hand prevents contact of components of the endoscope 60, like the shovel 402 and the multi-barreled cutting tool 300, with the tissue.

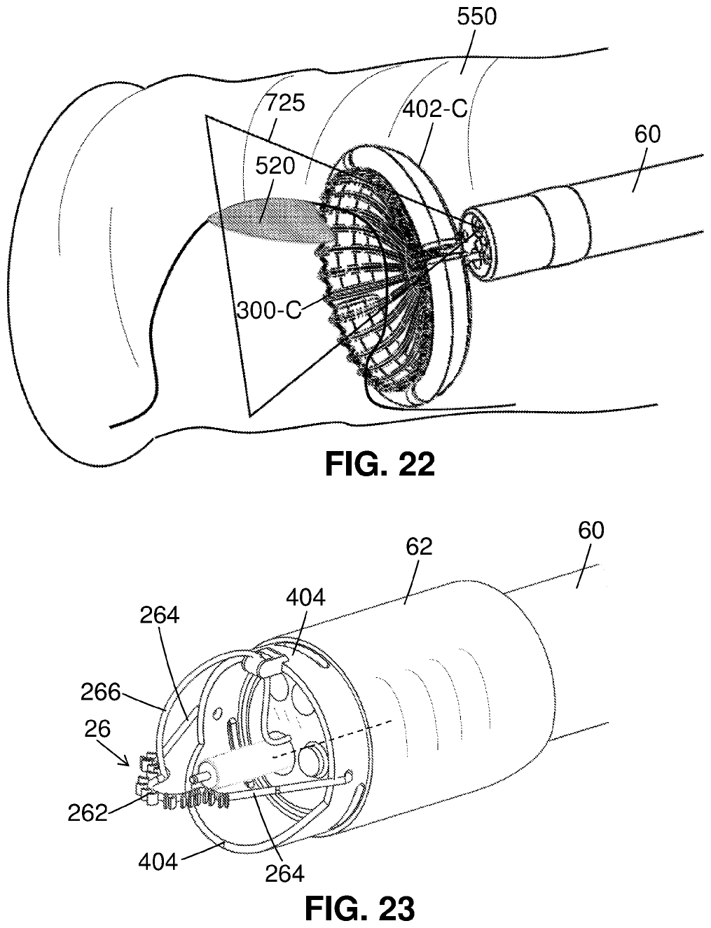

Referring now to FIG. 22, schematically illustrating, according to an exemplary embodiment, an endoscope comprising a circular shovel and a circular multi-barreled cutting tool, dissecting a target tissue. According to one embodiment, shown in FIG. 22, the shovel 402 is a circular shovel 402-C extending from a distal edge of the endoscope 60. According to another embodiment, a multi-barreled cutting tool 300 is attached to the circular shovel 402-C, thus rendering the multi-barreled cutting tool 300 a circular multi-barreled cutting tool 300-C. The circular shovel 402-C and the circular multi-barreled cutting tool 300-C enable dissection of a whole piece of target tissue 520 while keeping the endoscope 60 fixed in place, with a need to move the endoscope, or a prior art cutting tool 300 during the dissection of the target tissue 520. This is a great advantage over prior art endoscopes 60 that are not able to dissect a whole piece of target tissue 520 while keeping the endoscope 60 fixed in place.

As can be seen in FIG. 22, the endoscope 60 is inserted into a cavity 550, and the circular shovel 402-C extends out from the endoscope 60, while the circular multi-barreled cutting tool 300-C dissects an entire piece of a target tissue 520 while keeping the endoscope 60 fixed in place. An additional embodiment shown in FIG. 22 relates to a field of vision 725 of an imaging device of the endoscope 60. According to one embodiment, entire circular shove 302-C, and with it the entire circular multi-barreled cutting tool 300-C, is in the field of vision 725 of the imaging device of the endoscope 60. This embodiment allows visual inspection bu an operator of the dissection process of the entire piece of the target tissue 250 described above.

Referring now to FIG. 23, schematically illustrating, according to an exemplary embodiment, a side perspective view of an endoscope comprising a shovel and a lifting element. According to one embodiment, the shovel 402 further comprises a lifting element 26 configured to lift a target tissue 520, for example during manipulation of the target tissue 520. According to another embodiment, the lifting element 26 comprises a tissue engaging element 262 configured to engage with the target tissue 520 when lifting the target tissue 520. According to another embodiment, the tissue engaging element 262 has a high friction coefficient. This embodiment is important in order to allow tight engagement of the tissue engaging element 262 with the target tissue 520, and avoid slippage of the target tissue 520 from the tissue engaging element 262 during the lifting of the target tissue 520.

According to one embodiment, the lifting element 26 is configured to be in an elevated state and in a lowered state. Any mechanism for elevating and lowering the lifting element 26, and more particularly, elevating and lowering the tissue engaging element 262, is under the scope of the present subject matter. Such an exemplary mechanism is illustrated in FIG. 23. Thus, according to one embodiment, the lifting element 26 comprises at least one lifting arm 264, for example two lifting arms 264 as shown in FIG. 23, of which one side is attached to the tissue engaging element 262 and an opposite side is axially connected to the endoscope 60, or to the distal edge 62 of the endoscope, or to the connector 404 of the shovel 402. According to a further embodiment, the lifting element 26 is connected to an operator of the endoscope 60, either directly, or indirectly, for example via a motor, with a lifting cable 266. According to one embodiment, the lifting cable 266 is attached to the tissue engaging element 262, as shown in FIG. 23. According to another embodiment, the lifting cable 266 is attached to the at least one lifting arm 264.

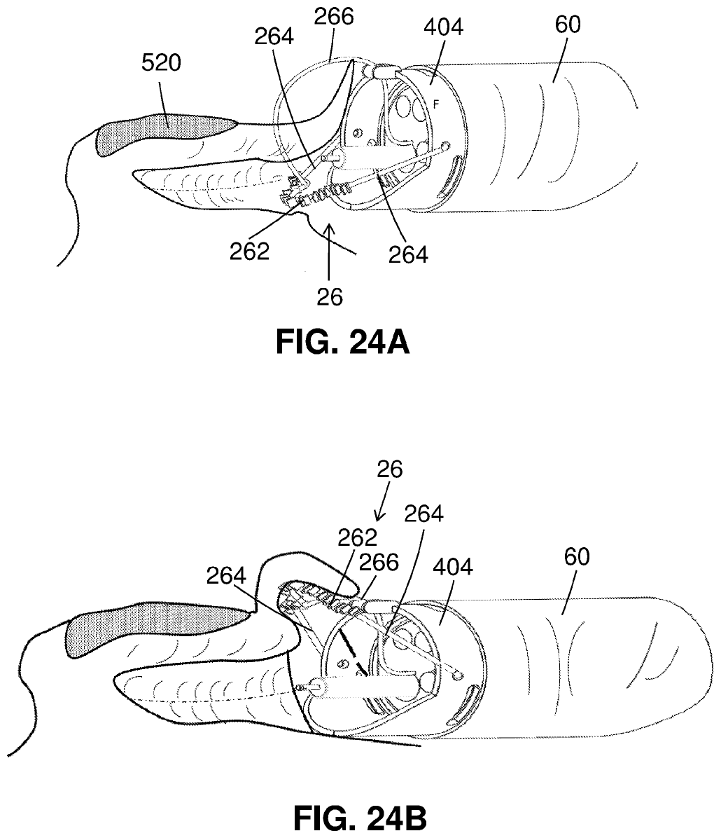

Referring now to FIGS. 24A-B, schematically illustrating, according to an exemplary embodiment, a side perspective view of an endoscope comprising a shovel and a lifting element in a lowered state and an elevated state lifting a target tissue, respectively. FIG. 24A shows the endoscope 60 positioned adjacent to a target tissue 520 when a cutting tool 300 forms a incision 522 in the target tissue 520 and as a result a part of the target tissue 520 lies over the shovel 402 and the connector 404. In addition, the lifting element 26 is in a lowered state, and the tissue engaging element 262 is positioned in the incision 522, under the part of the target tissue 520 that lies over the shovel 402 and the connector 404.

In FIG. 24B the lifting element 26 is in the elevated state. This can be achieved, for example, by pulling the lifting cable 266 that is attached to the tissue engaging element 262 according to the embodiment shown in FIGS. 23 and 24A-B. When the lifting element 26 is in the elevated state, the tissue engaging element 262 engages the target tissue 520, particularly the part of the target tissue 520 that lies over the shovel 402 and the connector 404, and lifts up the target tissue 520. The embodiment shown in FIG. 24B illustrates an advantage of the lifting element 26. By lifting the target tissue 520 there is a possibility to further dissect the target tissue with the cutting tool 300 and broaden the incision 522 further deeper in the target tissue 520. In addition, usage of the lifting element 26 allows removal of tissues that can block the movement of the endoscope 60 inside the cavity 550.

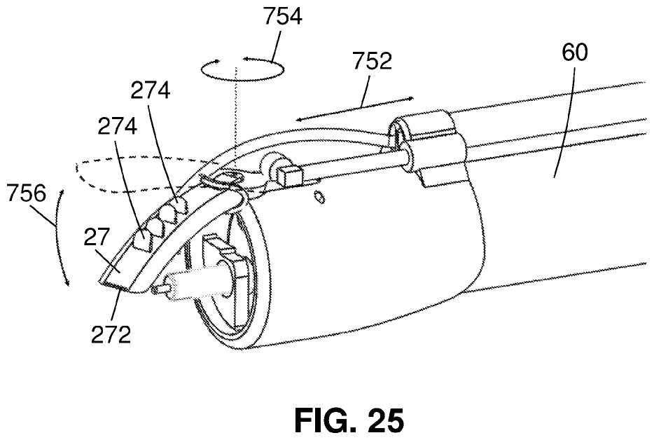

Referring now to FIG. 25, schematically illustrating, according to an exemplary embodiment, a side perspective view of an endoscope and a shovel comprising a sucking lifting element. According to one embodiment, the shovel 402 comprises a sucking lifting element 27 configured to engage with a target tissue 520 by a sucking force and lift or move aside the engaged target tissue 520. According to one embodiment, the sucking lifting element 27 has a nozzle-like structure that is attached to the endoscope 60. According to another embodiment, the sucking lifting element is attached to a shovel cover 80, or to a connector 404 of the shovel 404. Even though these embodiments are not shown in FIG. 25, they can be easily understood from FIG. 25.

According to one embodiment, the sucking lifting element 27 is configured to move, or tilt, in various directions, designated with arrows in FIG. 25. Arrow 752 designates that the sucking lifting element 27 is configured to move forward and backward along the length of the endoscope 60. Arrow 754 designates that the sucking lifting element 27 is configured to turn to the left and right in relation to the length of the endoscope 60. Arrow 756 designates that the sucking lifting element 27 is configured to move upwards and downwards. This versatility of the movement abilities of the sucking lifting element 27 provides a lot of freedom in engaging the target tissue 520 from different positions, and in addition provides a lot of freedom in the movement of the target tissue 520 after engagement with the sucking lifting element 27.

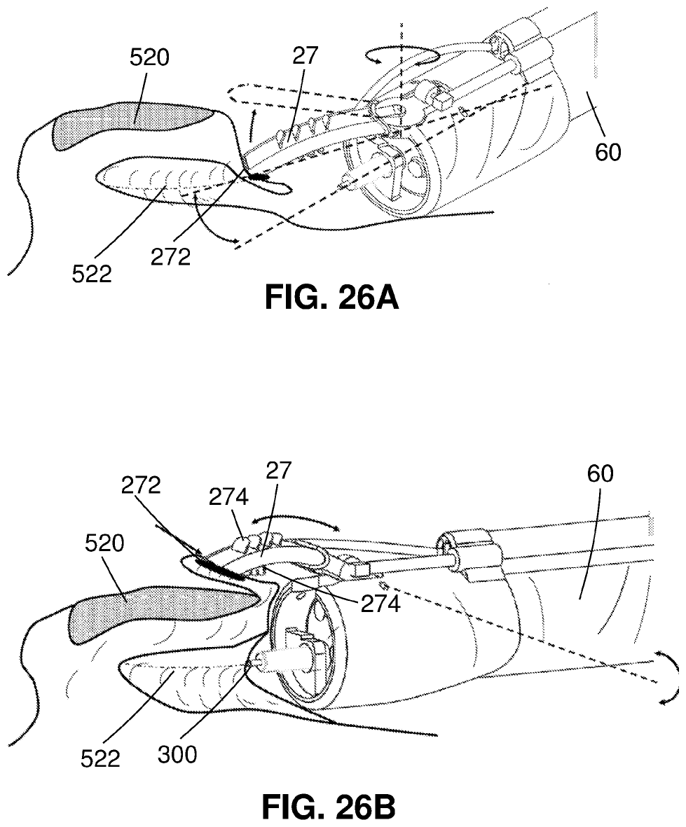

According to one embodiment, the sucking lifting element 27 comprises a tissue sucking element 272, positioned at a distal end of the nozzle-like structure of the sucking lifting element 27. The sucking lifting element 27 is hollow, and the tissue sucking element 272 is an opening at the distal end of the sucking lifting element. The tissue sucking element 272 is configured to engage with the target tissue 520, and then a sucking action in the sucking lifting clement 27 causes a formation of a vacuum force in the tissue sucking element 27, causing the target tissue 520 to be held by the sucking lifting element 27. When there is a desire to remove the target tissue 520 from the tissue sucking element 272, the sucking action though the sucking lifting element 27 is terminated, as a result the vacuum force in the tissue sucking element 272 dissipates, and the target tissue 520 is remove from the tissue sucking element 272. Illustrations of these embodiments are given in FIGS. 26A-B below.

According to one embodiment, the sucking lifting element 27 further comprises at least one bulge 274 on a top side of the sucking lifting element 27. The at least one bulge 274 allows engagement with the target tissue 520 in a manner similar to the engagement of the tissue engaging element 262 described above in FIGS. 23 and 24A-B.

Referring now to FIGS. 26A-B, schematically illustrating, according to an exemplary embodiment, a side perspective view of an endoscope comprising a sucking lifting element engaging with a target tissue, at a lowered state and an elevated state, respectively. FIG. 26A illustrates an endoscope 60 positioned in a vicinity of a target tissue 520, while dissecting the target tissue 520 with a cutting tool 300, thus forming an incision 522 in the target tissue 520. In addition, a sucking lifting element 27 is lowered down, while the tissue sucking element 272 engages with the target tissue 520.

In FIG. 26B, after the target tissue 520 is sucked by the tissue sucking element 272, the sucking lifting element 27 elevates to an elevated position. This allow further deepening the incision 522 in the target tissue 520 by the cutting tool 300. It should be noted that even though FIG. 26B only shown elevation of the target tissue 520 that is sucked by the sucking lifting element 27, it should be understood that the sucking lifting element 27 is configured to move in any desired direction, as described in FIG. 25, and therefore the sucked target tissue 520 can also be move in any desired direction.

Other embodiments relate to the at least one bulge 274. According to one embodiment, the at least one bulge 274 is positioned on a top side of the sucking lifting element 27, as shown in FIG. 25. According to another embodiment, the at least one bulge 274 is positioned on a bottom side of the sucking lifting element 27, as shown in FIG. 26B. According to yet another embodiment, the at least one bulge 274 is positioned on a top side and on a bottom side of the sucking lifting element 27.

According to one embodiment, the at least one bulge 274 has a high friction coefficient and is configured to engage with a target tissue 520 in a manner that keeps the target tissue engaged with the at least one bulge 274, without slippage of the target tissue 520 from the at least one bulge 274. According to another embodiment, the at least one bulge 275 is made of a material having on the surface a high friction coefficient, for example silicon, or a material similar to silicone.

A demonstration of an advantage of the at least one bulge 274 in the removal of the target tissue 520 with the sucking lifting element 27 is shown in FIG. 26B. FIG. 26B shows a sucking lifting element 27 engaging with the target tissue 520 by suction through the tissue sucking element 272. The engagement with the target tissue 520 take place inside an incision 522 made in the target tissue 520. Then, during lifting of the target tissue 520 by lifting the sucking lifting element 27, a part of the target tissue 520 engages with the at least one bulge 274 positioned on the bottom side of the sucking lifting element 27. Since the target tissue remains engaged with the at least one bulge 274 it is possible to lift the target tissue 520 and fold it upwards, as well as pushing it forward, for example to give access to the cutting tool 300, for example, for further deepening the incision 522.

The present subject matter additionally provides some improvements to a system for allowing access of a tool to all sides of an elevated tissue 520. Here is a general description of the system for allowing access of a tool to all sides of an elevated tissue, followed by a detailed description of some improvements in this system. For the sake of simplicity only, the system for allowing access of a tool to all sides of an elevated tissue 520, may be occasionally termed hereinafter “system 1”.

According to one embodiment, the system 1 is configured to allow access of various tools to all sides of the elevated tissue 520. Some exemplary tools include: a dissecting tool, a grabbing tool, an imaging tool, an injecting tool, a burning tool, and the like.

In some embodiments, the system 1 of the present subject matter allows dissection and separation of an elevated tissue 520 from a surface tissue 510 in a body of a patient. In some other embodiments, the system 1 of the present subject matter further allows removal of the dissected and separated elevated tissue 520 from the body of the patient.

According to one embodiment, the patient is an animal, particularly a vertebrate. According to another embodiment, the animal is a human.

The term “tool” as disclosed herein refers to any type of tool that is configured to be used during manipulation of tissues in a body of a patient. Some exemplary types of tools include; a dissecting tool configured to dissect a tissue; a grabbing tool configured to grab a piece of tissue; a storing tool configured to store an object, for example a piece of tissue, for example during removal of the object from the body of the patient; an imaging tool configured to acquire images inside a body of a patient; an illuminating tool configured to illuminate inside a body of a patient; an injecting tool configured to inject substances into a tissue; a burning tool configured to burn parts of a tissue, a combination thereof, and the like.

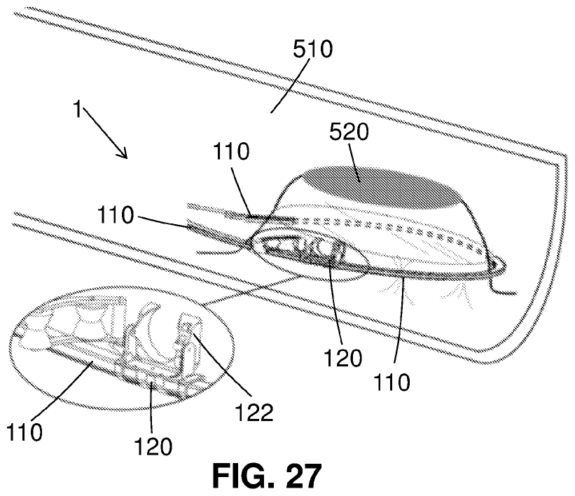

Referring now to FIG. 27, schematically illustrating, according to an exemplary embodiment, a system for allowing controlled access of a tool to all sides of an elevated tissue in a body of a patient. FIG. 27 illustrates components of a system 1 for allowing controlled access of a tool to all sides of an elevated tissue 520 in a body of a patient, the system 1 comprising:
a rail 110 configured to surround an elevated tissue 520 inside a body of a patient; and
at least one vehicle 120 configured to move along the rail 110 and carry at least one tool configured to manipulate the elevated tissue.

According to one embodiment, at least one connector 122 is attached to the vehicle 120, and configured to connect the at least one tool to the vehicle 120.

According to one embodiment, the tool is an integral part of the vehicle 120. According to another embodiment, the tool is separated from the vehicle and configured to connect to the vehicle 120.

As can be seen in FIG. 27, the surface tissue 510 is a part of a cavity in a body of a patient, and the elevated tissue 520 extends from the surface tissue 510. The rail 110 surrounds the elevated tissue 520, and the vehicle 120 is stilled attached to, or moves along, the rail 110. Since the rail 110 surrounds the elevated tissue 520, and the vehicle 120 is configured to move along the rail 110, the vehicle 120 can surround the elevated tissue 520. Thus, the system 1 allows access of the vehicle 120 to at least part of the elevated tissue 520, up to all sides of the elevated tissue 520, thereby allowing manipulation of the elevated tissue 520, depending on the tool connected to the vehicle 120.

Also illustrated in the circled zoom-in image in FIG. 27, at least one connector 122 attached to the vehicle 120. Thus, the system 1 allows access of any tool connected to the connector 122 to at least part of the elevated tissue 520, up to all sides of the elevated tissue 520.

According to one embodiment, the rail 110 and the vehicle 120 are configured to be inserted into a body of a patient. According to another embodiment, the rail 110 and the vehicle 120 are configured to be inserted into a cavity in the body of the patient. According to yet another embodiment, the rail 110 and the vehicle 120 are configured to be manually inserted into the body of the patient, or into a cavity in the body of the patient. According to a further embodiment, the rail 110 is configured to be inserted into the body of the patient, or into the cavity in the body of the patient, through an endoscope. According to yet a further embodiment, the vehicle 120 is configured to be inserted into the body of the patient, or into the cavity in the body of the patient, through an endoscope. According to still a further embodiment, the rail 110 and the vehicle 120 are both configured to be inserted into the body of the patient, or into the cavity in the body of the patient, through an endoscope. According to a further embodiment, the insertion of the rail 110, or the vehicle 120, or the rail 110 and the vehicle 120 can be either manual, or autonomous, namely by a robotic mechanism. According to an additional embodiment, the rail 110, or the vehicle 120, or both the rail 110 and the vehicle 120, are configured to be inserted in the body of the patient, or into the cavity in the body of the patient, through a multi-lumen that is transferred through an endoscope.

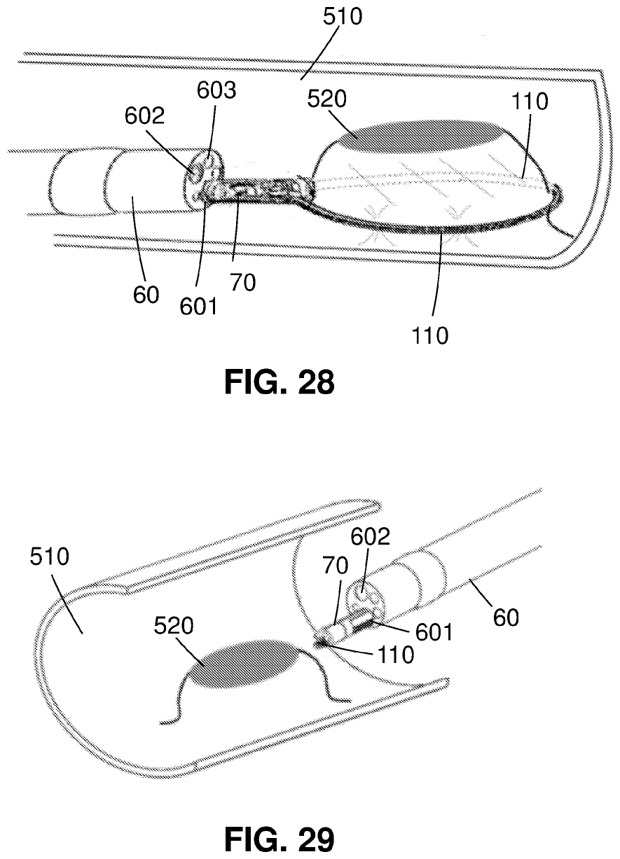

Referring now to FIG. 28, schematically illustrating, according to an exemplary embodiment, a rail surrounding an elevated tissue, the rail protrudes from an endoscope inserted into a cavity of a body of a patient. FIG. 28 illustrates an endoscope 60 that was inserted into a cavity of a body of a patient. Thus, the surface tissue 510 is the tissue of the cavity. As can be seen in FIG. 28, the endoscope 60 has a tube-like structure. The endoscope 60 comprises at least one, but preferably a plurality of channels 601, 602, 603, through which a rail 110, tools, or a combination of a rail 110 and tools, can be transferred. Further seen in FIG. 28 is a rail 110 exiting a channel 601 of the endoscope 60 and surrounding an elevated tissue 520 extending above the surface tissue 510. In other words, FIG. 28 illustrates the exemplary embodiment of a rail 110 configured to be inserted into a body of a patient, or into a cavity in the body of the patient, through an endoscope 60, for example via a multi-lumen 70. In this embodiment, during insertion of the endoscope 60 into the body of the patient the rail 110 resides inside a channel 601 of the multi-lumen 70 that is inserted in an endoscope 60. When the endoscope 60 approaches a vicinity of the elevated tissue 520, the multi-lumen 70 can depart the endoscope, as seen in FIG. 29, hereinafter, and the rail 110 can exit the channel 601 in which the rail 110 resides, and surround the elevated tissue 520.

Referring now to FIG. 29, schematically illustrating, according to an exemplary embodiment, a rail exiting a multi-lumen approaching an elevated tissue. FIG. 29 illustrates an internal cavity having a surface tissue 510, and an elevated tissue 520 extending above the surface tissue 510. Also shown is a multi-lumen 70 that protrudes from the endoscope 60 and approaching to the vicinity of the elevated tissue 520. During insertion of the endoscope 60 into the cavity, the multi-lumen 70 resides inside the working channel 601 of the endoscope 60, and the rail 110 resides in the multi-lumen 70 which is inside the working channel 601 of the endoscope 60. When the endoscope 60 approaches the vicinity of the elevated tissue 520, the rail 110 can exit from the channel 601 of the multi-lumen 70 towards the elevated tissue. FIG. 29 illustrates an edge of the rail 110 exiting the channel 601 of the endoscope 60. After the rail 110 exits the channel 601, the rail 110 is configured to surround the elevated tissue 520. It should be noted that hereinafter it will be mentioned that the rail 110 resides inside the endoscope 60. This description includes all the embodiments of storage of objects in the endoscope, like a working channel 601, a multi-lumen 70, and the like.

It should be noted again that the insertion of the rail 110 into the body to the vicinity of the elevated tissue 520, by using an endoscope 60, with or without a multi-lumen 70 in the endoscope 60, is only exemplary and should not be considered as limiting the scope of the present matter. The rail 110 can be brought to the vicinity of the elevated tissue 520 by any other mechanism as well, for example manually during an open surgery, or by any other means, for example a robotic arm, forceps, a combination thereof, and the like.

According to one embodiment, the system 1 is disintegrable. A capsule containing at least one component of the system 1 can be transferred to a target site in the patient's body by an endoscope, disintegrate from the endoscope and be left at the target site. Then, the component that is stored in capsules, for example a rail, a vehicle, a tool attached to the vehicle, and the like, can exit the capsule and operate at the target site.

Following are some improvements of the rail 110.

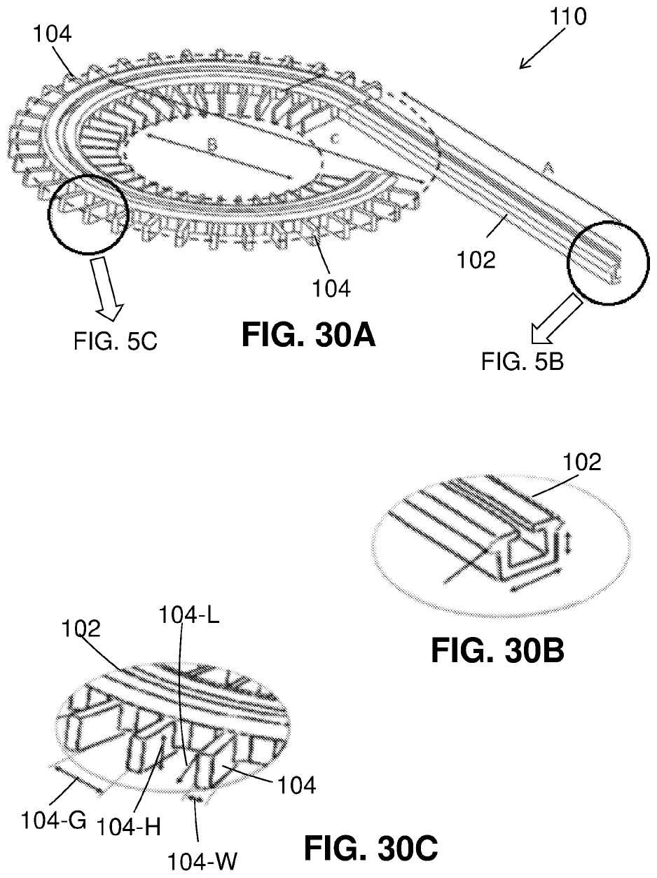

Referring now to FIG. 30A, schematically illustrating, according to an exemplary embodiment, a perspective view of a rail comprising a track and multiple ties extending from the track. FIG. 30A illustrates a rail 110 comprising a track 102, and multiple ties 104 extending from the track 102. According to one embodiment, the track 102 is configured to allow movement of a vehicle 120 along the track 102. In other words, the vehicle 120 is configured to be in contact with the track 102 and move along the track 102.

Referring now to FIG. 30B, schematically illustrating, according to an exemplary embodiment, an enlarged view of the track shown in FIG. 30A. According to one embodiment, shown in FIG. 30B, the track 102 can have a substantially U-shape profile that is configured to be in contact with the vehicle 120 and allow movement of the vehicle 120 along the track 102.

Returning now to FIG. 30A. The rail 110 shown in FIG. 30A is in a spread-out state, when most of the rail 110 is substantially circularly folded. In this state, the substantially circularly folded part of the rail 110 is configured to surround an elevated tissue 520, essentially as shown in FIGS. 2 and 3. According to one embodiment, the substantially circularly folded state of the rail 110 is a default state of the rail 110. In other words, the rail 110 always tends to be in the substantially circularly folded state. When the rail 110 is stored inside an endoscope 60, the rail 110 is forced to be in a shape that is determined by the shape of the endoscope 60, for example a linear shape, because is it forced to adapt to the shape of the endoscope 60. However, when the rail 110 is pushed out of the endoscope 60, for example in a vicinity of an elevated tissue 520, as shown in FIG. 29, that shows the rail 110 in an early stage of exiting the endoscope 60, the rail 110 tends to return to its default substantially circularly folded state, and thereby surround the elevated tissue 520.

Still referring to FIG. 30A. As mentioned above, the rail 110 comprises multiple tics 104 extending from the track 102. According to one embodiment, the ties 104 extend substantially perpendicularly relative to the track 102. According to another embodiment, the ties 104 extend from the part of the track 102 that is configured to be in a substantially circularly folded state, namely at the part of the track 102 that is configured to surround the elevated tissue 520. According to yet another embodiment, the ties 104 extend from both sides of the track 102. In other words, there are ties 104 that extend toward the inner circle of the rail 110, toward the elevated tissue 520 that is surrounded by the rail 110; and there are ties 104 that extend out of the circle of the rail 110, away from the elevated tissue 520 that is surrounded by the rail 110.

According to one embodiment, the ties 104 are configured to stabilize the rail 110 when the rail 110 surrounds an elevated tissue 520. This embodiment is achieved by increasing the area of contact of the rail 110 with the tissue by the ties 102. In addition, the individual ties 104 that are in contact with the tissue are somewhat anchored in the tissue, and thus further stabilized the rail 110 in place. According to another embodiment, the ties 104 are configured not to harm the tissue that they are in contact with. For example, edges of the ties are blunt so as to not puncture and injure the tissue. Another example can be that the ties 104 are elastic in a manner that causes the ties 104 to bend when they press the tissue.

Referring now to FIG. 30C, schematically illustrating, according to an exemplary embodiment, an enlarged view of the track and ties shown in FIG. 30A. FIG. 30A illustrates some dimensions of ties 104. The tie 104 has a length 104-L that is the distance between the track 102 from which the tic 104 extends and a distal edge 104-E of the tic 104; a width 104-W as seen in FIG. 30C; a height 104-H as seen in FIG. 30C, and between adjacent ties 104 there is a gap 104-G. According to one embodiment, the dimensions of the ties 104, together with the type of material of which the rail 110 is made, determine the level of elasticity and rigidity of the rail 110, particularly in the part of rail 110 that has a default substantially circularly folded state.

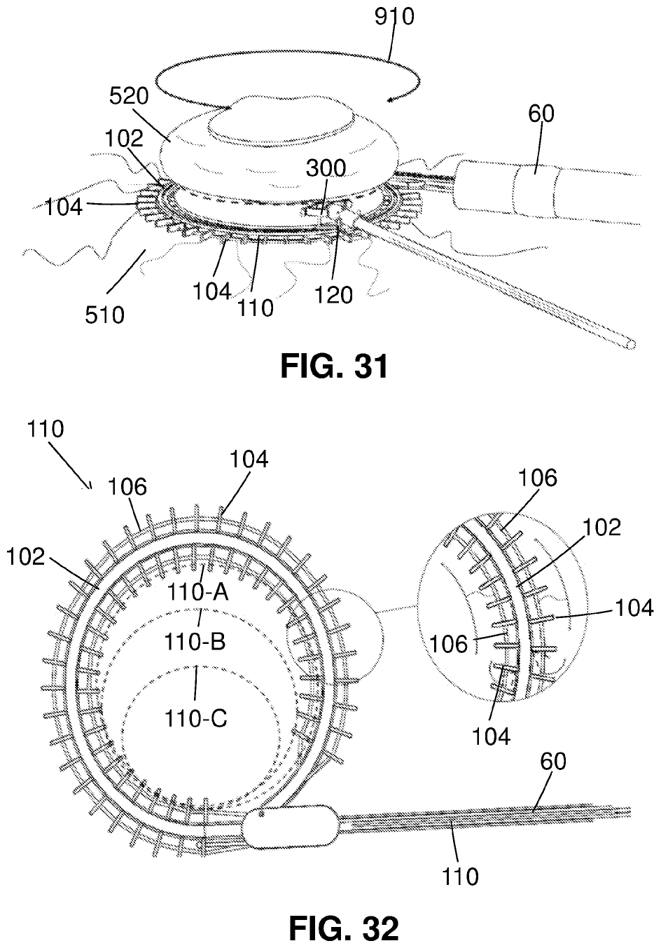

Referring now to FIG. 31, schematically illustrating, according to an exemplary embodiment, a perspective view of a rail comprising a track and multiple ties extending from the track exiting from an endoscope and surrounding an elevated tissue, while a vehicle stands on, or moves along, the rail and cuts the elevated tissue. FIG. 31 shows a rail 110 exiting an endoscope 60 and surrounding an elevated tissue 520. It is well shown how the ties 104 of the rail 110 are in contact with the surface tissue 510 and the elevated tissue 520, thus stabilizing the rail 110 in place. Further shown in FIG. 31 is a vehicle 120 standing on, or moving along, the rail 110, while cutting the elevated tissue. Circular arrow 910 represents an exemplary path 910 of the vehicle 120 along the rail 110. Broken line 521 represents a pre-determined, or pre-planned, cutting line 521 of the elevated tissue 520, that is to be cut by a cutting tool attached to the vehicle 120.

In experiments conducted with the rail 110 shown in FIG. 27, that does not comprise ties 104, and a vehicle 120 with a cutting tool 300 attached to the vehicle 120, it was found that the elevated tissue 520, especially a soft elevated tissue 520, tends to collapse over the rail 110 and block movement of the vehicle 120 along the rail 110, thus disturbing the cutting of the elevated tissue. However, in similar experiments conducted with the rail 110 comprising a track 102 and multiple ties 104 extending from the track 104, shown in FIG. 31, it was found that the ties 104 remove the elevated tissue 520 from the track 102, thus allowing free movement of the vehicle 120 along the rail 110, and undisturbed cutting of the elevated tissue 520. Thus, according to one embodiment, the ties 104 are configured to remove the tissue from the rail 110 and allow undisturbed movement of the vehicle 120 along the rail 110. More particularly, the ties 104 are configured to remove a tissue that is at both sides of the rail 110—the side that is in contact with the elevated tissue 520, and the ties 104 that extend away from the elevated tissue 520. This clears the way at both sides of the rail 110, for smooth movement of the vehicle 120 along the rail 110.

Another feature of the ties 104 is that the ties 104 increase the area of contact of the rail 110 with the tissue, and thereby increasing the stability of the rail 110. High stability of the rail 110 is especially important during the manipulation of the elevated tissue 520, for example during cutting of the elevated tissue 520.

In addition, it was found that the ties 104 facilitate formation of an elevated tissue having a symmetrical structure, also when the elevated tissue is asymmetric in nature. A symmetric structure of the elevated tissue 520 is advantageous because it allows more accurate cutting of the elevated tissue 520. Additionally, it was found that the ties 104 facilitate a formation of an elevated tissue 520 having a clear mushroom-like shape, which is a preferable shape of a target tissue in the art of dissection of tissues in a patient.

Furthermore, it was found that the ties 104 create a linear path on the tissue, also in cases where the tissue is not planar, but rather folded, or not straight in nature, for example in the stomach or the intestine. This linear path is advantageous, because it provides the vehicle 120 a linear path for smooth movement, even on a folded tissue.

Referring now to FIG. 32, schematically illustrating, according to an exemplary embodiment, an upper view of a rail comprising a track and multiple ties extending from the track, exiting an endoscope, and having various stages of extension. FIG. 32 illustrates a rail 110 exiting an endoscope 60, and assuming the default substantially circular state as described above. During the process of cutting of an elevated tissue 520 by a cutting tool 300 that is attached to a vehicle 120 that runs along the rail 110, the diameter of the elevated tissue 520 at the cutting line 521 (see FIG. 31) is reduced. Therefore, the diameter of the circle that is formed by the rail 110 has to be reduced accordingly in order to allow the cutting tool 300 to continue to be in contact with the elevated tissue 520 at the cutting line 521, in order to further cut the elevated tissue 520. This is achieved by pulling the rail 110 back into the endoscope 60 during the cutting of the elevated tissue 520, in order to reduce the diameter of the circled rail 110.

The pulling of the rail 110 back into the endoscope 60 during the cutting of the elevated tissue 520 is executed by an operator of the system 1. According to one embodiment, the operator can get a visual image of the dissected elevated tissue 520 during the cutting, by a camera of the endoscope 60, and according to the image decide when there is a need to pull the rail 110 in order to tighten its grasping of the elevated tissue 520, and to what extent. For example, the operator can compare between the visual image of the elevated tissue and pre-planned images of the stages of the operation in order to determine when and how to pull the rail 110. The operator can also determine the distance between the border to the elevated tissue 520 and the track 102, or the ties 104. When this distance is higher than a certain pre-determined value, there is a need to pull the rail 110 until the distance is close enough. In addition, the operator can see the level of coverage of the ties 104 by the elevated tissue 520. When the ties 104 are at least partially covered by the elevated tissue 520, this indicates that the rail 110 is close enough to the elevated tissue 520 and there is no need to pull the rail 110. On the other hand, when the ties 104 are exposed, this indicates that the rail 110 is distant from the elevated tissue 520 and there is a need to pull the rail 520 in order to tighten the grasping of the elevated tissue 520 by the rail 110.

According to another embodiment, pressure sensors, or contact sensors, can be attached to the rail 110, for example to the ties 104. When a pressure is detected by the pressure sensors, or when a contact is detected by the contact sensors, this indicates that the ties 104 are covered by the elevated tissue 520, and there is no need to pull the rail 110. On the other hand, when a lower pressure on the pressure sensors is sensed, or when no contact is detected by the contact sensors, this indicates that the rail is disconnected from the elevated tissue 520, and there is a need to pull the rail 110 in order to bring the rail 110 to a closer proximity to the elevated tissue 520.

According to yet another embodiment, the operator can sense the force that he uses to pull the rail 110. When the rail 110 is distant from the elevated tissue 520, there is a lower resistance to the pulling force exerted on the rail 110. This indicates to the operator that there is a need to further pull the rail 110 until the rail 110 get in contact with the elevated tissue 520, and the resistance to the pulling force exerted on the rail 110 increases.

Broken lines 110-A, 110-B, and 110-C represent various positions of the rail 110 during the process of cutting an elevated tissue 520. Line 110-A represents an initial state, when the rail 110 just surrounds and elevates the elevated tissue 520 Line 110-B represents an intermediate state, when the elevated tissue 520 is partially cut, and the diameter of the cutting line 521 of the elevated tissue 520 is reduced. At this stage, the rail 110 is partially pulled into the endoscope 60, and as a result the circle of the rail 110 corresponds now broken line 110-B. Line 110-C represents a final state, when the elevated tissue 520 is almost completely cut, and the diameter of the cutting line 521 of the elevated tissue 520 is further reduced. At this stage, the rail 110 is further pulled into the endoscope 60, and as a result the circle of the rail 110 corresponds now to broken line 110-C. Also shown in FIG. 32, is the part of the rail 110 that is pulled back into the endoscope 60, showing the endoscope 60 in a transparent view.

Still referring to FIG. 32. According to one embodiment, the ties 104 are foldable and can be in two states: an extending state, in which the ties extend substantially perpendicularly to the track 102; and a folded state, in which the ties 104 are folded and are parallel to the track 102, and essentially tightly attached to the track 102. According to another optional embodiment, the extending state is the default state of the ties 104. In other words, the ties 104 naturally tend to extend from the track 102. When the rail 110 is free, namely, not inside an endoscope 60, the ties 104 are extended. As a result, the ties 104 assume the folded state when they are forced to do so, for example when the rail 110 resides inside the endoscope 60. Any mechanism that allows changing of the state of the ties, between the extending state and the folded state, is under the scope of the present subject matter. Some exemplary mechanisms include: the ties 104 are pivotally attached to the track 102; the track 102 and ties 104 are made of a material having a shape memory that is designed such that the default state of the ties 104 is the extending state, and the like. Another mechanism involves the usage of a string 106, as follows:

Still referring to FIG. 32, the ties of the rail 110 that extend to the same side of the track 104 are connected by at least one string 106. As can be seen in FIG. 32, the ties 104 that extend toward the inner circle of the rail 110, or toward the elevated tissue 520 surrounded by the rail 110, are connected with a string 106; and also the ties 104 that extend outwardly from the circle, or away from the elevated tissue 520, are connected with a string 106. It should be noted though, that according to some other embodiments, only the ties 104 the extend toward the elevated tissue 520 are connected with a string 106, or only the ties 104 that extend away from the elevated tissue 520 are connected with a string 106.

According to one embodiment, the string 106 passes through the endoscope 60, and is operable by a user, or operator of the rail 110. In other words, pushing or pulling the string 106 is carried out by a user or an operator of the rail 110. For example, the string 106 can be pulled in order to bring the ties 104 from the extending state to the folded state, or pushed in order to bring the ties 104 from the folded state to the extending state. The string 106 is useful also in an embodiment in which the ties 104 have a shape memory of the extending state. In this embodiment, the string 106 can be pulled in order to force the extending ties 104 to fold, or the string 106 can be released in order to allow ties 104 to return to the default extending state.

According to one embodiment, the rail 110 has, in general, three states: a storage state, in which the rail 110 resides inside an endoscope 60, when the shape of the rail 110 is defined by the shape of the endoscope 60; a deployment state in which the rail 110 exits the endoscope 60, assumes the default substantially circularly folded state, and thereby encircles an elevated tissue; a working state in which the elevated tissue 520 is treated by a tool attached to a vehicle 120 that moves along the rail 110-when the tool is a cutting tool 300, the rail 110 is gradually pulled back into the endoscope 60, thereby decreasing the diameter of the circle of the rail 110; and a completion state, after the elevated tissue 520 has been treated, in which the entire rail 110 is pulled back into the endoscope 60, while being forced to assume a shape similar to the shape of the endoscope 60. The completion state essentially returns the rail 110 back to the storage state.

Following are some improvements of the tool:

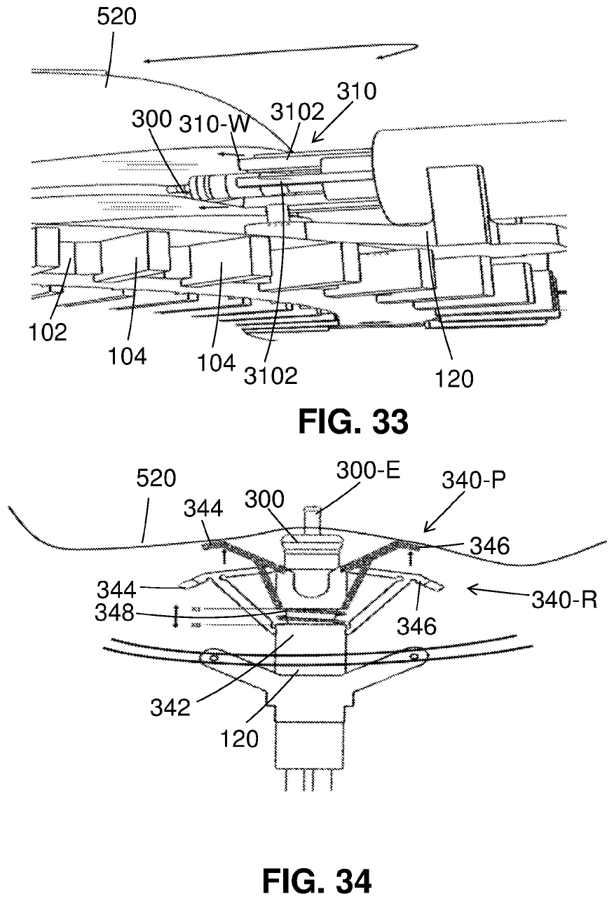

Referring now to FIG. 33, schematically illustrating, according to an exemplary embodiment, a side view of a tool comprising a wrap. FIG. 33 shows a rail 110 comprising a track 102 and ties 104, as described above, a vehicle 120 moving along, or standing on, the rail 110, and a tool, for example a cutting tool 300, attached to the vehicle 120. In FIG. 33, the cutting tool 300 cuts an elevated tissue 520 that is surrounded by the rail 110. In experiments conducted with a cutting tool 300, as is, without any additional component, it was found that during cutting the elevated tissue tends to collapse on the cutting tool 300, thus blocking the movement of the vehicle 120 that carries the cutting tool 300, blocking insertion of the cutting tool 300 inside the dissected elevated tissue 520 for further cutting the elevated tissue 520, and also causing bending of the cutting tool 300, in cases where the cutting tool 300 is not rigid enough. A warp 310 that wraps the cutting tool 300, at least partially, overcomes the aforementioned challenges.

According to one embodiment, the cutting tool 300 comprises a wrap 310 that wraps the cutting tool 300 at least partially. According to another embodiment, the wrap 310 is configured to support the cutting tool 300 and prevent bending of the cutting tool 300. According to yet another embodiment, the wrap 310 is configured to remove the collapsing tissue 520 from the cutting tool 300, thus allowing the cutting tool 300 to freely cut the elevated tissue 520, and also allowing undisturbed movement of the vehicle 120 carrying the cutting tool 300 along the rail 110.

According to one embodiment, shown in FIG. 33, the wrap 310 is made of a plurality of ribs 3102 arranged around at least part of cutting tool 300, and parallel to the cutting tool 300, when there is a gap between adjacent ribs 3102. According to another embodiment, the wrap 310 is an intact cylinder that at least partially wraps the cutting tool 300.

According to one embodiment, the wrap 310 has a width 310-W. According to another embodiment, the width 310-W of the wrap 310 is big enough to efficiently remove the collapsing elevated tissue 520 from the cutting tool 300. For example, the width 310-W of the wrap 310 is substantially 1.5 mm.

According to one embodiment, an edge of the wrap 310 that is in contact with the collapsed elevated tissue 520 is straight as can be seen in FIG. 33. Even though the edge of the wrap 310 can have any other shape, for example a rounded shape, the straight shape of the edge is preferable in terms of efficiently removing the collapsed elevated tissue 520 from the cutting tool 300.

Referring now to FIG. 34, schematically illustrating, according to an exemplary embodiment, an upper view of a cutting tool comprising a dynamic tissue remover. Another means to remove a collapsed elevated tissue 520 from the cutting tool 300 is by using the dynamic tissue remover 340 that is shown in FIG. 34. According to one embodiment, the dynamic tissue remover 340 comprises a slider 342 attached to the cutting tool 300 and configured to slide along the cutting tool 300, a first arm 344 pivotally attached to one side of the slider 342, and a second arm 346 pivotally attached to an opposite side of the slider 342. FIG. 34 shows two states of the dynamic tissue remover 340. The dynamic tissue remover 340 that is drawn in white is in a resting state 340-R, and the dynamic tissue remover 340 that is drawn in black is in a pushing state 340-P. In the resting state 340-R, the slider 342 is positioned distantly from an edge 300-E of the cutting tool 300, and the first arm 344 and the second arm 346 are pulled away from the elevated tissue 520 that is cut by the cutting tool 300. At the resting state 340-R there is no removal of tissue from the cutting tool 300. In order to bring the dynamic tissue remover 340 to the pushing state 340-P. the slider 342 is pushed toward the edge 300-E of the cutting tool 300. As a result, the first arm 344 and the second arm 366 are pushed toward the elevated tissue 520, and as a result remove the elevated tissue 520 from the edge 300-E of the cutting tool 300, thereby allowing undisturbed cutting of the elevated tissue 520, and free movement of the vehicle 120 that carries the cutting tool 300.

According to one embodiment, the dynamic tissue remover 340 additionally comprises an elastic member 348, for example a spring 348, that renders one of the states of the dynamic tissue remover 340 as default. According to one embodiment, the spring 348 renders the resting state 340-R as default. According to another embodiment, the spring 348 renders the pushing state 340-P as default. When the dynamic tissue remover 340 is brought to a corresponding state that is not default, when released, the dynamic tissue remover 340 returns autonomously to the default state.

There is provided a system for allowing controlled access of a tool to all sides of an elevated tissue in a body of a patient, the system comprising:
a rail configured to surround the elevated tissue; and
at least one vehicle configured to move along the rail and carry at least one tool that is configured to manipulate the elevated tissue,
wherein the rail comprises a track and multiple ties extending from the track.

According to one embodiment, the patient is an animal.

According to one embodiment, the animal is a vertebrate.

According to one embodiment, the animal is a human.

There is also provided a system for allowing controlled access of a cutting tool to all sides of an elevated tissue in a body of a patient, the system comprising:
a rail configured to surround the elevated tissue; and
at least one vehicle configured to move along the rail and carry at least one cutting tool comprising a blade configured to cut the elevated tissue.

According to one embodiment, the patient is an animal.

According to one embodiment, the animal is a vertebrate.

According to one embodiment, wherein the animal is a human.

According to one embodiment, the cutting tool comprises a wrap that wraps the cutting tool at least partially.

According to one embodiment, the wrap is made of a plurality of ribs arranged around at least part of cutting tool, and parallel to the cutting tool, when there is a gap between adjacent ribs.

According to one embodiment, the wrap is an intact cylinder that at least partially wraps the cutting tool.

According to one embodiment, the cutting tool comprises a dynamic tissue remover.

There is additionally provided a system for allowing controlled access of a tool to all sides of an elevated tissue in a body of a patient as herein described with reference to the accompanying drawings.

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A shovel configured to be attached to an endoscope having a distal edge, wherein the shovel is configured to protrude from a lower part of the distal edge, and wherein the shovel further comprising a shovel rail that runs along a width of the shovel and is substantially vertical relative to a length of the endoscope, wherein the shovel rail is configured to allow a vehicle to move along the shovel rail, thus allowing the vehicle to move along a width of the shovel.

2. (canceled)

3. The shovel of claim 1, having a substantially concave structure.

4. The shovel of claim 1, further comprising a connector configured to connect the shovel to the distal edge of the endoscope.

5-6. (canceled)

7. The shovel of claim 1, further comprising a shovel cover comprising a covering element configured to cover the shovel from above and allow protrusion of a tool out from the shovel.

8. The shovel according to claim 7, wherein the shovel cover comprises an opening at a front side relative to the distal edge of the endoscope, the opening being configured to allow protrusion of a tool therethrough.

9. The shovel of claim 7, wherein the shovel cover permanently covers the shovel.

10. The shovel of claim 7, wherein the shovel cover is removable.

11. The shovel of claim 7, wherein a cover connector is attached to the shovel cover and is configured to connect the shovel cover to the distal edge of the endoscope.

12. The shovel of claim 11, wherein the cover connector is in a form of a sleeve that engages with the distal edge of the endoscope.

13-14. (canceled)

15. The shovel of claim 7, wherein the covering element is at least partially foldable.

16. The shovel of claim 4, wherein the shovel is axially attached to the connector with a horizontal axis positioned at a bottom part of the connector, wherein the horizontal axis allows swiveling of the shovel either upwards, or downwards, or upwards and downwards.

17. The shovel of claim 4, wherein the shovel is axially attached to the connector with a vertical axis positioned at a border between the shovel and the connector, wherein the vertical axis allows swiveling of the shovel either to the right, or to the left, or to the right and to the left.

18. The shovel of claim 1, further comprising a swiveling sub-shovel positioned over, or under the shovel, and configured to swivel aside, thus allowing extension of the width of the shovel.

19. (canceled)

20. The shovel of claim 1, further comprising a lifting element configured to lift a target tissue, wherein the lifting element is configured to be in an elevated state and in a lowered state.

21. The shovel of claim 20, wherein the lifting element comprises a tissue engaging element configured to engage with the target tissue when lifting the target tissue.

22. (canceled)

23. The shovel of claim 1, further comprising a sucking lifting element configured to engage with a target tissue by a sucking force and lift or move aside the engaged target tissue, wherein the sucking lifting element has a nozzle-like structure.

24. The shovel of claim 23, wherein the sucking lifting element comprises a tissue sucking element positioned at a distal end of the nozzle-like structure of the sucking lifting element, wherein the sucking lifting element is hollow, and the tissue sucking element is an opening at the distal end of the sucking lifting element, wherein the tissue sucking element is configured to engage with the target tissue, and then a sucking action in the sucking lifting element causes a formation of a vacuum force in the tissue sucking element, causing the target tissue to be held by the sucking lifting element.

25. The shovel of claim 23, wherein the sucking lifting element further comprises at least one bulge on a top side of the sucking lifting element, or on a bottom side of the sucking lifting element, or on both the top side and the bottom side of the sucking lifting element, wherein the at least one bulge allows engagement with the target tissue.

26. An endoscope comprising the shovel of claim 1, attached to a distal edge of the endoscope.

27. The endoscope of claim 26, further comprising a gripper attached to a distal edge of the endoscope, wherein the gripper is configured to grip a target tissue in a vicinity of the shovel.
